(19) United States
(12) Patent Application Publication
(10) Pub. No.: US 2002/0034515 A1
(43) Pub. Date: Mar. 21, 2002

(54) Proteus mirabilis-based vaccine

(76) Inventors: Harry L.T. Mobley, Baltimore, MD (US); Xin Li, Elicott City, MD (US); John W. Warren, Baltimore, MD (US)

Correspondence Address: Lepold Presser, Esq., SCULLY, SCOTT, MURPHY & PRESSER, 400 Garden City Plaza, Garden City, NY, 11530, US

(21) Appl. No.: 09/862,810

(22) Filed: May 22, 2001

Related U.S. Application Data
(60) Provisional application No. 60/206,163, filed on May 22, 2000.

(51) Int. Cl. A61K 39/02; C07H 21/04; C12N 1/21; C07K 14/195; C12N 15/74

(52) U.S. Cl. 424/190.1; 530/350; 435/252.3; 435/69.3; 435/320.1; 536/23.7

(57) ABSTRACT

The present invention is directed to a gene encoding an adhesin polypeptide of Proteus mirabilis, the MrpH gene, its gene product, antigenic fragments of the adhesin polypeptide and antibodies directed against the MrpH gene product and fragments thereof. The present invention is also directed to a vaccine against Proteus mirabilis and treatment of Proteus mirabilis infection with anti-adhesin antibodies.

This invention was made with Government support under P01 DK 49720 awarded by the National Institutes of Health. The Government has certain rights in the invention.

### FIELD OF THE INVENTION

The present invention is directed to a gene encoding an adhesin polypeptide of Proteus mirabilis, the MrpH gene, its gene product and antibodies directed against the MrpH gene product. The present invention is also directed to a vaccine against Proteus mirabilis and treatment of Proteus mirabilis infection with anti-adhesin antibodies.

### BACKGROUND OF THE INVENTION

Proteus mirabilis is not a common cause of UTI in the normal host. Rubin, R. H. et al. (1986) Urinary tract infection, pyelonephritis, and reflux nephropathy. p. 1085-1141. In B. M Brenner and F. C. Rector (eds.), The Kidney. W. B. Saunders Co., Philadelphia. Surveys of uncomplicated cystitis or acute pyelonephritis show that P. mirabilis comprises only a few percent of cases. Even in patients with recurrent UTI, the incidence of infections by this organism is only a few percentage points higher. However, this organism infects much higher proportions of patients with complicated urinary tracts, i.e., those with functional or anatomic abnormalities or with chronic instrumentation such as long-term catheterization (see Ehrlich, O. and A. S. Brem. (1982) Pediatrics 70:665-669) and has a predilection for the kidney Fairley, K. F. et al. (1971) Lancet ii:615-618. In these patients, not only does this bacterium cause cystitis and acute pyelonephritis, (Eriksson, S., et al. (1986) Scand. J. Infect. Dis. 18:431-438; File, T., (1985) Am. J. Med. 79:91-95), but also the production of urinary stones, a hallmark of infection with this organism (Griffith, D. P. et al. (1976) Invest. Urol. 13:346-350.). The production of urinary stones adds another dimension to these already complicated urinary tracts.

Proteus mirabilis expresses several types of fimbrial structures that promote attachment to and colonization of host mucosal surfaces (Mobley et al. (1995) Trends Microbiol. 3:280-284; Mobley et al. (1994) Kidney Int. 46:S129-S136). One of these, mannose resistant Proteus-like (MR/P) fimbria, a surface structure responsible for mannose-resistant hemagglutination (MRHA), has been shown to contribute significantly to the development of experimental urinary tract infections (UTIs) (Mobley et al. (1990) J. Infect. Dis. 161:525-530).

P. mirabilis is a motile gram-negative bacterium. P. mirabilis expresses a number of proteins that have been correlated with virulence in the urinary tract. Such virulence factors include urease, two proteases, flagella, fimbriae (four distinct types: MR/P, PMF, ATF, and NAF), hemolysin, and amino acid deaminase. Urease, IgA protease, flagella, a 37-kDa protease, and MR/P fimbriae, contribute most significantly to the pathogenesis of acute pyelonephritis. PMF fimbriae and hemolysin also contribute to virulence, but play a more subtle role. The virulence factors are distinct from those described for E. coli. P. mirabilis, which can differentiate from short vegetative cells to elongated highly flagellated forms, is found in soil, water, and the human intestinal tract (Hoeniger, J. F. M. (1964) Can. J. Micro. 10: 1-9). Proteus was aptly named by Hauser in 1885 for the character in Homer's Odyssey who “has the power of assuming different shapes to escape being questioned (Hoeniger, J. F. M. (1965) J. Gen. Micro. 40:29-42; Hoeniger, J. F. M. (1966) Can. J. Micro. 12:113-122.).”

Stone formation is caused by the expression of a highly active urease which hydrolyzes urea to ammonia, causing local pH to rise with subsequent precipitation of magnesium ammonium phosphate (struvite) and calcium phosphate (apatite) crystals (Griffith, D. P., et al. (1976) Invest. Urol. 13:346-350;Mobley, H. L. T. et al. (1989) Microbiol. Rev. 53:85-108). The stones resulting from aggregation of such crystals complicate infection for three reasons. First, the P. mirabilis caught within the interstices of the forming stones are very difficult to clear with only antibiotics. Second, the stone is a nidus for non-P. mirabilis bacteria to establish UTI which also are difficult to eradicate. Third, the stone can obstruct urine flow; pelvic and renal stones are often associated with acute pyelonephritis, pyonephrosis, and/or chronic pyelonephritis.

Infection of the urinary tract by P. mirabilis is governed by the general principles of bacterial pathogenesis. Proteins produced by this species during infection contribute to a) colonization of the host; b) evasion of host defense; and c) damage of host tissue.

Colonization requires movement of P. mirabilis to its niche, attachment to specific receptors, and survival in the urinary tract. At least four fimbriae have been identified in this species (Adegbola, R. A., et al. (1983) J. Med. Microbiol. 16:427-43; Bahrani, F. K., et al. (1991) Infect. Immun. 59:3574-3580; Bahrani, F. K.,et al. (1993) Infect. Immun. 61:884-891; Massad, G., et al. (1994) Infect. Immun. 62:1989-1994.). MR/P (mannose-resistant Proteus-like) fimbriae and PMF (P. mirabilis fimbriae) contribute to colonization by P. mirabilis.

Proteus produces an amino acid deaminase, an enzyme which generates α-keto acids which can bind ferric iron (Fe³⁺) and allow growth of the bacterium in an otherwise iron-limiting environment (Drechsel, H., A. (1993) J. Bacteriol. 175:2727-2733).

To move to the site of colonization, P. mirabilis undergo swarmer cell differentiation (i.e., development of an elongated form on solid surfaces) which is accompanied by production of hundreds of flagella per cell. The flagella-mediated motility is required to ascend the ureters to the kidney. Allison et al. ((1992) Infect. Immun. 60:4740-4746.) have shown that the genes encoding flagellin, urease, and hemolysin are coordinately expressed in these differentiated cells. The expression of each of these virulence genes is very low in the normal vegetative cell but rises dramatically during swarmer cell differentiation. These data suggest that differentiation into swarmer cells is an adaptive response of P. mirabilis to the host environment.

P. mirabilis avoids host defenses by at least three mechanisms. These include production of an IgA-degrading protease (ZapA) that cleaves secretory IgA, produced in response to infection (Loomes, L. M., (1990) Infect. Immun. 58:1979-1985; Loomes, L. M.,(1992) Infect. Immun. 60:2267-2273). P. mirabilis has three distinct copies of flagellin genes that recombine and express antigenically novel flagellins. Bacteria expressing these recombinant flagellins thrive under the selective pressure of the host immune response (Belas, R., et al. (1994) Gene 148:33-41). Since the flagella represent major surface antigens for this species during infection, synthesis of antigenically distinct flagella could represent an effective mechanism for avoidance of host defense. Finally, expression of MR/P fimbriae can also be modulated by a mechanism of phase variation, resulting in expression of the fimbriae by some bacteria and no expression by other bacteria within the same population at a given time.

Hemolysin and urease appear to contribute to damage of host tissue (Chippendale, G. R., et al. (1994) Infect. Immun. 62:3115-3121.; Johnson, D. E.,et al. (1993b) Infect. Immun. 61:2748-2754; Jones, B. D., et al. (1990) Infect. Immun. 58:1120-1123.; Mobley, H. L. T., et al. (1991) Infect Immun. 59:2036-2042.). The HpmA hemolysin is a potent cytotoxin in vitro for renal epithelial cells, cytolyzing such primary human cell cultures after only brief contact (Mobley, H. L. T., et al. (1991) Infect Immun. 59:2036-2042.). Urease hydrolyzes urea in urine, present in high concentration [0.5 M], releasing ammonia and raising the local pH which also damages epithelium (Johnson, D. E., et al. (1993b) Infect. Immun. 61:2748-2754; Jones, B. D., et al. (1990) Infect. Immun. 58:1120-1123.). Stones formed due to urease can also physically damage the epithelium and block urine flow.

P. mirabilis is a virulent pathogen that is well adapted for life in the urinary tract. P. mirabilis contaminates the periurethral area and can colonize the bladder epithelium via PMF fimbriae that bind to specific (but unknown) receptors on bladder epithelium. Once established, motile flagellated bacteria ascend the ureters to the kidney. MR/P fimbriae then are able to bind specifically to renal epithelium (Bahrani, F. K., et al. (1994) Infect. Immun 62:3363-3371; Silverblatt, F. (1974) J. Exp. Med. 140:1696-171; Silverblatt, F. J. and I. Ofek. (1978) J. Infect. Dis. 138:664-667.) where they are expressed by >98% of the bacterial population in the kidney (Zhao, H., et al. (1997) Molec. Microbiol. 23:1009-1019.). As a consequence of binding, a biofilm microenvironment can be established (Lerner, S. P., et al. (1989) J. Urol. 141:753-757). During this process, urease hydrolyzes urea in the urine causing a rise in local pH which initiates precipitation of supersaturated polyvalent cations and anions in the form of struvite and apatite stones (i.e., urolithiasis). Epithelial cell necrosis or apoptosis may occur. Hemolysin, a potent in vitro cytotoxin, appears to play a role once the organism has reached the kidney parenchyma. In addition, bacteria may enter renal tubule epithelial cells with unknown consequence.

There is a need for safe, effective, long-acting vaccines against P. mirabilis infection. A number of antigen preparations have been tested. Legagno-Fajardo ((1991) Can. J. Microbiol. 37:325-328.) found that parenteral immunization with purified MR/P fimbriae protects mice from transurethral challenge with homologous and heterologous strains. An outer membrane protein preparation was used by O'Hanley's group ((1991) Infect. Immun. 59:3778-3786) to protect Balb/C mice from homologous intravesicular challenge. In addition, Braude ((1969) J. Immunol. 102:454-465.) immunized rats parenterally with purified flagella. Braude demonstrated that antiserum immobilized bacteria by binding to flagella and prevented the organisms from spreading from one kidney to the other kidney (i.e., down one ureter, into the bladder, and back up the other ureter). None of the tested antigen preparations relate to components of the MR/P fimbriae, nor are such preparations adapted for intranasal administration. In addition, the tested antigen preparations are generally acknowledged as being inadequate in spectrum and duration of protection. Thus, the present discovery provides new vaccines for treating or preventing P. mirabilis-mediated diseases including, but not limited to, urolithiasis, acute pyelonephritis, and bacteremia. The present discovery also provides vaccines for preventing the development of bladder and kidney stones in humans, having significant advantages over those of the prior art.

### SUMMARY OF THE INVENTION

The present invention is directed to isolated DNA encoding Proteus mirabilis adhesin polypeptide (MrpH). The MrpH polypeptide is encoded by nucleotides 8126-8953 of the MrpH gene, as shown in FIG. 1 (SEQ ID NOS: 1&2). The MrpH polypeptides, and antigenic peptide fragments thereof, are useful in vaccines against P. mirabilis and for the production of antibodies directed against the MrpH polypeptide.

Another aspect of the present invention provides an isolated nucleic acid encoding an MrpH polypeptide as describe above, or a fragment thereof, and replicable expression vectors containing these nucleic acids.

A still further aspect of this invention is directed to transformed hosts such as prokaryotic microorganisms and cultured eukaryotic cells containing the replicable expression vectors encoding MrpH polypeptides.

Another aspect of this invention provides isolated MrpH protein and antigenic MrpH peptides in recombinant form.

A further aspect of this invention provides a vaccine composition for immunization against P. mirabilis containing a MrpH polypeptide or antigenic portions thereof and a pharmaceutically acceptable carrier.

A still further aspect of this invention provides polyclonal and monoclonal antibodies directed against the MrpH, hybridoma cell lines producing these monoclonal antibodies and methods of using these antibodies to detect P. mirabilis infection in vertebrates, including humans.

Yet another aspect of this invention is directed to a method of treatment or prevention of P. mirabilis infection by the administration of antibodies directed against the MrpH protein, including treatment by passive immunization.

Still yet another aspect of the present invention is directed to pharmaceutical compositions comprising MrpH polypeptides and a pharmaceutically acceptable carrier.

### BRIEF DESCRIPTION OF THE DRAWINGS

FIG. 1A illustrates the Proteus mirablils fimbrial operon.

FIG. 1B illustrates the nucleotide sequence of the Proteus mirablils fimbrial operon, strain HI4320

FIG. 2 illustrates the nucleic acid encoding MrpH polypeptide.

FIG. 3 illustrates the polypeptide sequence of MrpH.

FIG. 4 The amino acid sequences of MrpH, SmfG, and PapG were aligned using GCG “pileup” command. Gaps (.) were introduced to obtain maximal fit. The amino acid residues that are conserved in two or more of the proteins are noted at the bottom of the alignment. Amino acid residues that are conserved in all three are highlighted in black boxes.

FIG. 5 The amino acid sequences of the six putative pilins of MR/P fimbria were aligned using GCG “pileup” command. Gaps (.) were introduced to obtain maximal fit. The amino acid residues that are conserved in three or more of the pilins are noted at the bottom of the alignment. The amino acid residues that are conserved in all six pilins are highlighted in black boxes. *, the valine residue and the isoleucine residue are both conserved in three of the six pilins. Aligned with the other putative MR/P pilins, MrpH is the only one that contains a distinctive N-terminal domain that could provide a possible basis for the receptor-binding activity.

FIG. 6 The predicted restriction map of the wild type strain and mrpH::aphA mutant is illustrated in the top panel. The arrows represent predicted open reading frames within the defined region. The size of the bands that hybridized with the probes in the Southern blots are indicated along the left side.

FIG. 7A is a scattergraph showing intranasal immunization with MrpH conjugated to cholera toxin reduced the number of bacteria in the kidneys and bladders of immunized mice by >80-fold compared to a naive controls.

FIG. 7B is a scattergraph showing intranasal immunization with MrpH conjugated to cholera toxin reduced the number of bacteria in the urine, kidneys and bladders of immunized mice by >80-fold compared to a naive controls.

FIG. 8A. Independent challenge experiment: a group of 12 mice and a group of 10 mice were transurethrally challenged with 4.8×10⁶ CFU/mouse of the wild type strain and 5.5×10⁶ CFU/mouse of the mrpH::aphA mutant, respectively.

FIG. 8B. Co-challenge experiment: a group of 10 mice was transurethrally challenged with 1:1 mixture of the wild type strain and the mrpH::aphA mutant (7.9×106 CFU of the wild type strain and 1.2×10⁷ CFU of the mrpH: :aphA mutant per each mouse). After 7 days, mice were sacrificed and quantitative bacterial counts in urine, bladders, and kidneys were calculated. Each diamond represents the CFU/ml urine or CFU/g tissue from an individual mouse. Horizontal bars represent the geometric means of the colony counts. The range of detection in this assay is 10² to 10⁹ CFU/ml urine or CFU/g tissue. Values ≦10² were set to 10², and values ≧10⁹ were set to 10⁹. P values (bottom) were derived using an unpaired, one-tailed Mann-Whitney test. Lf., left; Rt., right. WT, the wild type strain; mrpH, the mrpH::aphA mutant.

FIG. 9A is a bar graph showing protection of kidneys by FimH-and MrpH-Adhesin-based vaccines.

FIG. 9B is a bar graph showing protection of bladder by FimH-and MrpH-Adhesin-based vaccines.

### DETAILED DESCRIPTION OF THE INVENTION

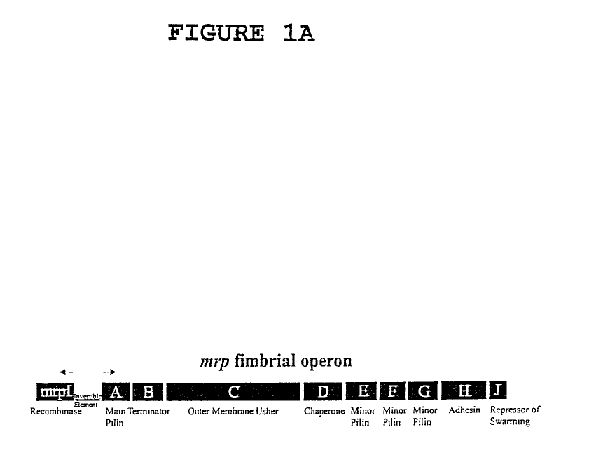

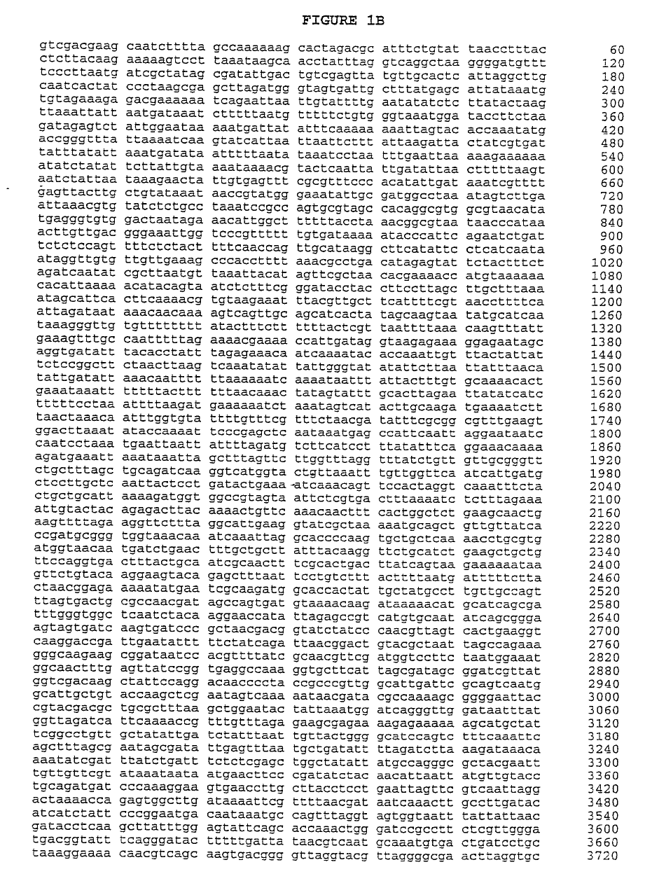

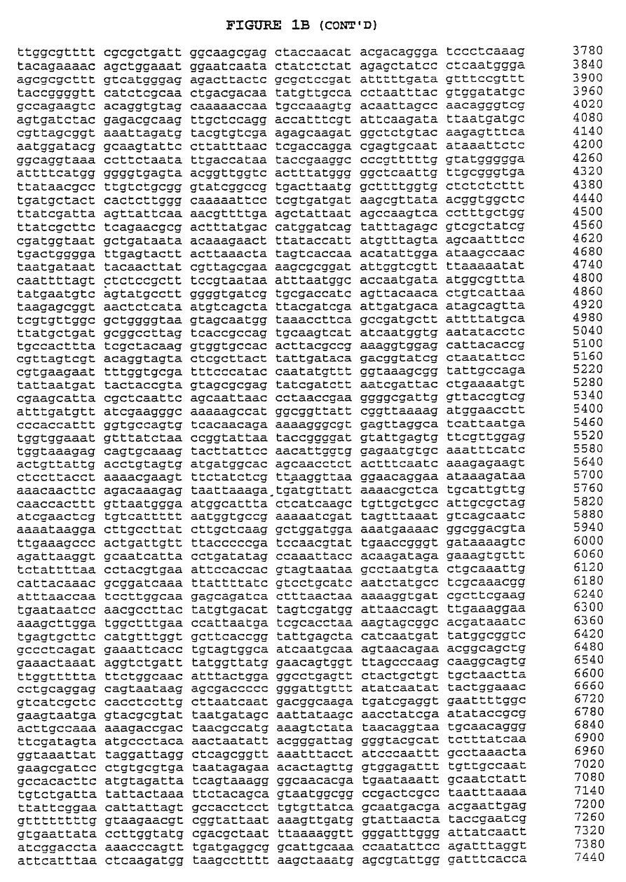

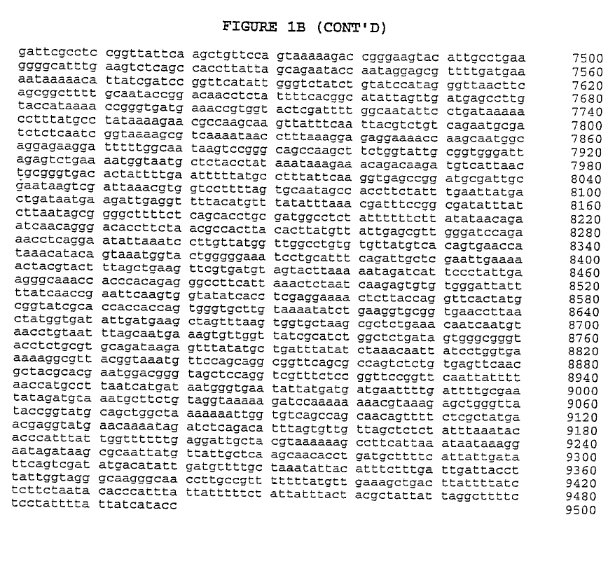

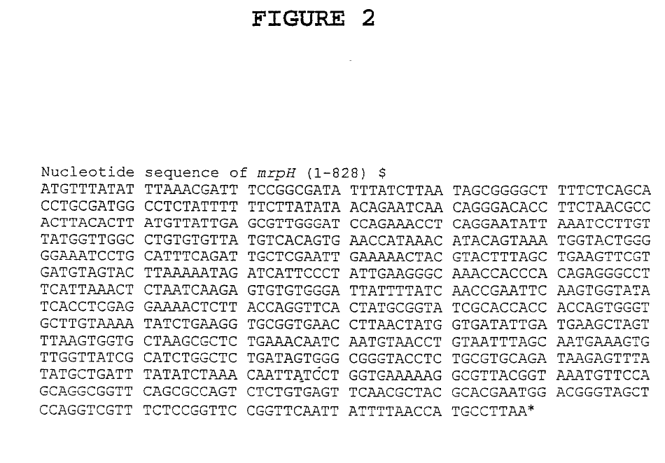

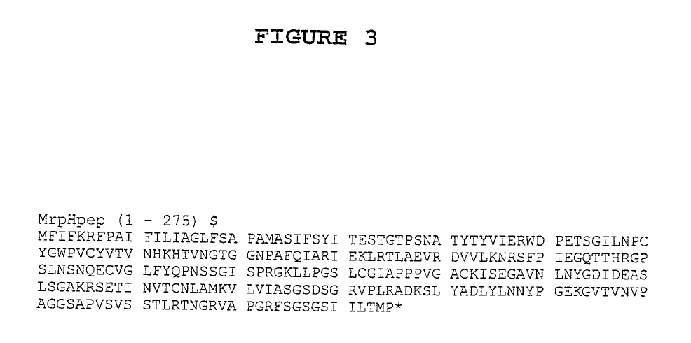

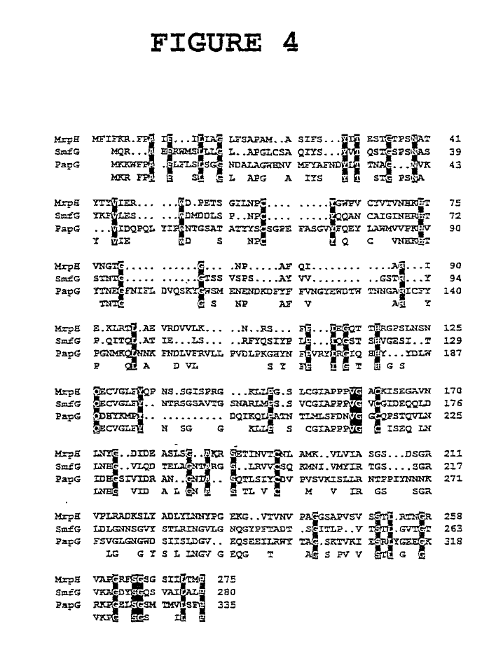

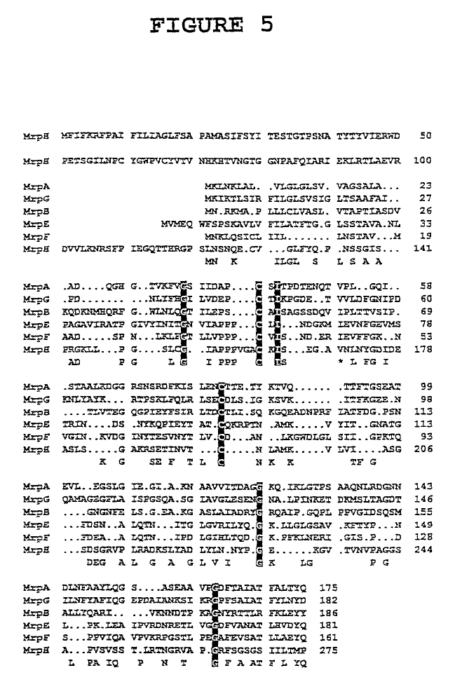

The present invention provides the Proteus mirabilis adhesin polypeptide gene (MrpH), encoded by the nucleotide sequence in FIG. 1 (nucleotides 8126-8953 of FIG. 1) (SEQ ID NOS: 1 & 2). The MrpH gene is 828 nucleotides (FIG. 2, SEQ ID NO: 3) and encodes a polypeptide of 275 amino acid residues (FIG. 3, SEQ ID NO:2). The amino acid sequence of MrpH shares 30% identity with PapG and 35% identity with SmfG (FIG. 4, SEQ ID NOS: 4 & 5), both of which were demonstrated to be fimbrial adhesins (Lindberg, F., et al. (1987) Nature 328:84-87; Mizunoe, Y., et al. (1991) J. Bacteriol. 173:3257-3260). Protein sequence alignment of the six putative pilins of MR/P fimbria (MrpA (SEQ ID NO: 6); MrpG (SEQ ID NO: 7); MrpB (SEQ ID NO: 8); MrpE (SEQ ID NO: 9); MrpF (SEQ ID NO: 10); FIG. 5) show that all of the pilins except MrpH were similar in size and share amino acid sequence identity throughout their entire predicted amino acid sequence, especially at the C-terminal chaperone-binding sequence (Gly and Tyr residues are the 14th and 2nd amino acid residue from the C-terminus, respectively). Aligned with the other putative MR/P pilins, MrpH has a distinctive N-terminus, a putative region for receptor-binding activity. The C-terminal chaperone-binding domain of MrpH is unique in that it does not retain the conserved Tyr residue at the penultimate position. Also, MrpH has a Pro residue at the last position, a feature that has been conserved in many fimbrial adhesins from different species including PapG, PrsG, SmfG, Fim2, and Fim3 (FIG. 4). Based on the sequence homology, we discovered that MrpH was the functional MR/P hemagglutinin. The present invention provides a complete DNA sequence for the MrpH adhesin gene and allows identification of the MrpH adhesin polypeptides encoded thereby.

Another aspect of the present invention provides replicable expression vectors allowing regulated expression of a Proteus mirabilis adhesin polypeptide. Replicable expression vectors as described herein are generally DNA molecules engineered for controlled expression of a desired gene, especially high level expression where it is desirable to produce large quantities of a particular gene product, or polypeptide. The vectors encode promoters and other sequences to control expression of the gene being expressed, and an origin of replication which is operable in the contemplated host. Preferably the vectors are plasmids, bacteriophages, cosmids or viruses. Any expression vector comprising RNA is also contemplated.

Sequence elements capable of effecting expression of a gene product include promoters, enhancer elements, transcription termination signals and polyadenylation sites. Promoters are DNA sequence elements for controlling gene expression, in particular, promoters specify transcription initiation sites. Prokaryotic promoters that are useful include the mrp promoter, the lac promoter, the trp promoter, and PL and PR promoters of lambda and the T7 polymerase promoter. Eukaryotic promoters are especially useful in the invention and include promoters of viral origin, such as the baculovirus polyhedrin promoter, the vaccinia virus hemagglutinim (HA) promoter, SV40 late promoter, the Moloney Leukemia Virus LTR, and the Murine Sarcoma Virus (MSV) LTR. Yeast promoters and any promoters or variations of promoters designed to control gene expression, including genetically-engineered promoters are also contemplated. Control of gene expression includes the ability to regulate a gene both positively and negatively (i.e., turning gene expression on or off) to obtain the desired level of expression.

One skilled in the art has available many choices of replicable expression vectors, compatible hosts and well-known methods for making and using the vectors. Recombinant DNA methods are found in any of the myriad of standard laboratory manuals on genetic engineering (see for example Sambrook, et al., 1989, Molecular Clonging: A Laboratory Approach, 2^{nd} edition, Cold Sring Harbor Laboratory Press, Cold Spring Harbor, N.Y.).

The replicable expression vectors of the present invention can be made by ligating part or all of the MrpH coding region in the proper orientation to the promoter and other sequence elements being used to control gene expression. For example, a DNA fragment encoding MrpH nucleotides 8126-8953 may be operably linked, by ligation, downstream of a promoter, thereby allowing expression of a 275 amino acid MrpH polypeptide. This juxtapositioning of promoter and other sequence elements with the MrpH polypeptide coding region allows the production of large amounts of the MrpH polypeptide useful, not only as a vaccine against Proteus mirabilis infection, but also for anti-MrpH antibody production and for analysis of the function of MrpH during Proteus mirabilis infection.

Preferred vectors of the present invention are derived from eukaryotic sources. Expression vectors that function in tissue culture cells are especially useful, but yeast vectors are also contemplated. These vectors include yeast plasmids and minichromosomes, retrovirus vectors, BPV (bovine papilloma virus) vectors, baculovirus vectors, SV40 based vectors, vectors and other viral vectors. Baculovirus vectors and retrovirus vectors (e.g., murine leukemia viral vectors) are preferred. Tissue culture cells that are used with eukaryotic replicable expression vectors include S. frugiperda cells, VERO cells, MRC-5 cells, SCV-1 cells, COS-1 cells, NIH3T3 cells, mouse L cells, HeLa cells and such other cultured cell lines known to one skilled in the art.

The present invention also contemplates prokaryotic vectors that are suitable as cloning vectors or as expression vectors for MrpH polypeptides, including bacterial and bacteriophage vectors that can transform such hosts as E. coli, B. subtilis, Streptomyces sps. and other microorganisms. Many of these vectors are based on pBR322 including pUC19, pGEM-72f and pRK2 (commercially available from Promega, Madison, Wis.) and are well known in the art. Bacteriophage vectors that are used in the invention include lambda and M13.

In another embodiment, the adhesin protein of the present invention is expressed in a baculovirus expression system. This system provides baculovirus expression vectors into which MrpH DNA encoding an MrpH polypeptide can be inserted downstream of a strongly transcribed promoter. When cultured in insect cells, the recombinant baculovirus can provide stable expression of high levels of extracellular or intracellular polypeptide. Baculovirus expression vectors and their use are reviewed in Luckow et al. ((1988) Bio. Technology 67:47-55). A particular advantage of this system is its similarity to higher eukaryotes with regard to protein modification, processing and transport. Thus, recombinant-derived eukaryotic proteins will be processed and glycosylated in a manner important for obtaining a native protein conformation and, hence, maximal biological activity.

The vectors of the present invention may also contain other sequence elements to facilitate vector propagation, isolation and subcloning; for example, selectable marker genes and origins of replication that allow for propagation and selection in bacteria and host cells. Selectable marker genes can include ampicillin and tetracycline resistance genes for propagation in bacteria or neomycin or zeocin resistance for selection in mammalian cells. In addition, the vectors of the present invention may comprise a sequence of nucleotides for one or more restriction endonuclease sites. Use of nucleic acid fragments having multiple cloning sites are also contemplated by the present invention as are reporter genes. Coding sequences for heterologous genes, sequences for selectable markers, reporter genes and multiple cloning sites are well known to the skilled artisan. Examples of reporter genes include GFP, luciferase, CAT, and -galactosidase. By “heterologous genes” is meant coding sequences or parts thereof which are not derived from MprH.

The choice of vectors may vary, depending on the host cells. When the host cell is a eukaryotic cell, yeast, viral or baculoviral vectors are preferred. When the host cell is a prokaryotic cell, then phage vectors are preferred, such as a phage vector. The resulting vector-carried library is then transformed into a host cell.

The skilled artisan is familiar with the choice of host cells, including bacteria cells such as strains of E. coli, strains of Pseudomonas such as Pseudomonas aeruginosa or strains of Bacillus, yeast cells such as strains of Saccharomyces or Pichia pastoris, CHO cells such as CHO-1, COS cells such as COS-7 and insect host cells such as Spodoptera frugiperda. Once the host cells receive the vectors, the cells are cultivated and induced so as to express the proteins encoded by the nucleic acid molecules of the cDNA library.

A further aspect of the present invention is directed to an isolated MrpH polypeptide, especially a recombinant MrpH polypeptide and fragments thereof. A MrpH polypeptide can be obtained from cultured cells, from infected animals or from microorganisms or cells transformed with an expression vector encoding a MrpH polypeptide. A process of preparing a recombinant MrpH polypeptide includes cultivating the microorganism or cell transformed with an recombinant nucleic acid for a time and under conditions sufficient to produce a MrpH polypeptide and then recovering the MrpH polypeptide. Purification of a MrpH polypeptide is achieved by conventional purification techniques such as ammonium sulfate precipitation, column chromatography, affinity chromatography and the like. During purification, the MrpH polypeptide is identified by SDS-polyacrylamide gel electrophoresis, or by standard immunodetection techniques, such as immunoblotting or immunoprecipitation.

According to the present invention a MrpH “fragment” refers to any subject peptide having an amino acid sequence shorter than that of the adhesin protein depicted in FIG. 3. The fragments of the present invention are derived from the full-length MrpH protein and retain the antigen specificity of the instant MrpH protein. For example, a MrpH peptide fragment contemplated by the present invention is (MrpH 23-157) MASIFSYI TESTGTPSNA TYTYVIERWD PETSGILNPC YGWPVCYVTV NHKHTVNGTG GNPAFQIARI EKLRTLAEVR DVVLKNRSFP IEGQTTHRGP SLNSNQECVG LFYQPNSSGI SPRGKLLPGS LCGIAPP (SEQ ID NO: 11). Preferably, the fragment contains at least about 20 contiguous amino acids and has the antigenicity of the protein of FIG. 3. The fragment can also contain at least about 8 contiguous amino acids having the antigenic activity of the protein of FIG. 3. Preferably, the fragment is derived from MrpH amino acids 23-157 of SEQ ID NO: 11.

Antibodies can be used to purify an MrpH polypeptide or fragments thereof. Antibodies are highly specific and are especially useful for isolating specific antigens (proteins) that represent only minor components of complex mixtures such as cell lysates. The lambda gtll expression system (Sambrook et al. (1989) Molecular Cloning: A Laboratory Manual Vol. 2, Cold Spring Harbor Laboratory Press 12.1-12.44) provides a fusion protein of beta-galactosidase and MrpH proteins. This fusion protein can be purified by passage of a cell lysate containing the fusion protein over an anti-beta-galactosidase immuno-affinity column. The anti-beta-galactosidase antibodies bound to the column matrix bind the fusion protein. Any impurities can be washed off the column and the fusion protein can be diluted by changes in pH, or by use of detergents, chaotropic agents or organic solvents. Immunoaffinity purification techniques are well known in the art (see, for example, Harlowe, et al., 1988, Antibodies: A Laboratory Manual, Cold Spring Harbor Laboratory Press: 511-552). The purified MrpH fusion protein can be used to obtain antibodies specific for the MrpH protein. These anti-MrpH antibodies in turn allow immunoaffinity purification of the non-fusion MrpH protein or peptides thereof.

Another embodiment of the present invention provides polyclonal antibodies directed against the MrpH protein or peptides encoding a portion of an MrpH polypeptide. The antibodies are useful for passive immunization of mammals infected with Proteus mirabilis and for the purification of MrpH polypeptides. Antibodies can be generated by using an entire adhesin polypeptide as an antigen or by using short peptides encoding a fragment or portion of an adhesin polypeptide, such as MrpH, as antigens.

The following peptide is a preferred immunogen for generating antibodies:

Peptide 1 (MrpH 23-157) MASIFSYI TESTGTPSNA TYTYVIERWD PETSGILNPC YGWPVCYVTV NHKHTVNGTG GNPAFQIARI EKLRTLAEVR DWLKNRSFP IEGQTTHRGP SLNSNQECVG LFYQPNSSGI SPRGKLLPGS LCGIAPP (SEQ ID NO: 11)

Polyclonal antibodies directed against an MrpH polypeptide or antigenic peptide thereof are prepared by injection of a suitable animal with an immunogenic amount of the peptide or antigenic component, collecting serum from the animal, and testing sea for the desired reactivity. If necessary, specific sera can be isolated by any of the known immunoadsorbent techniques. Detailed protocols for antibody production are provided in Harlow, E. et al. Antibodies: A Laboratory Manual, Cold Spring Harbor Press, New York, (1988).

Another embodiment of the present invention provides monoclonal antibodies. Monoclonal antibodies are preferred because large quantities of antibodies, all of similar reactivity, are produced. The preparation of hybridoma cell lines for monclonal antibody-production is done by fusing an immortal cell line with antibody-producing lymphocytes from an immunized animal. This can be done by techniques which are well known to those who are skilled in the art. (See, for example, Harlow, E. and Lane, D., Antibodies: A Laboratory Manual, Cold Spring Harbor Press, (1988); or Douillard, J. Y. and Hoffman, T., “Basic Facts About Hydridomas”, in Compendium of Immunology Vol. II, L. Schwartz (Ed.), (1981).

Functional derivatives of the monoclonal antibodies of the present invention are also contemplated. “Functional derivatives” refer to antibody molecules or fragments thereof that are derived from the instant monoclonal antibodies and that have retained the antigen specificity of the instant monoclonal antibodies. Examples of functional derivatives include Fab, Fab′, F(ab′)₂ of the present mAbs, single chain antibodies, humanized antibodies and the like.

A single-chain antibody (SAb) is created by fusing together the variable domains of the heavy and light chains using a short peptide linker, thereby reconstituting an antigen binding site on a single molecule. Such single-chain antibody variable fragments (Fvs) can be fused to all or a portion of the constant domains of the heavy chain of an immunoglobulin molecule, if necessary. The use of sAb avoids the technical difficulties in the introduction of more than one gene construct into host cells. Single chain antibodies and methods for their production are known in the art. See, e.g., Bedzyk et al. (1990) J. Biol. Chem., 265:18615; Chaudhary et al. (1990) Proc. Natl. Acad. Sci., 87:9491; U.S. Pat. No. 4,946,778 to Ladner et al.; and U.S. Pat. No. 5,359,046 to Capon et al.

The monoclonal antibodies of the present invention can be humanized to reduce the immunogenicity for use in humans. For example, to humanize a monoclonal antibody raised in mice, one approach is to make mouse-human chimeric antibodies having the original variable region of the murine mAb, joined to constant regions of a human immunoglobulin. Chimeric antibodies and methods for their production are known in the art. See, e.g., Cabilly et al., European Patent Application 125023 (published Nov. 14, 1984); Taniguchi et al., European patent Application 171496 (published Feb. 19, 1985); Morrison et al., European Patent Application 173494 (published Mar. 5, 1986); Neuberger et al., PCT Application WO 86/01533, (published Mar. 13, 1986); Kudo et al., European Patent Application 184187 (published Jun. 11, 1986); Robinson et al., International Patent Publication #PCT/US86/02269 (published May 7, 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988). These references are incorporated herein by reference. Generally, DNA segments encoding the H and L chain antigen-binding regions of the murine mAb can be cloned from the mAb-producing hybridoma cells, which can then be joined to DNA segments encoding CHand CL regions of a human immunoglobulin, respectively, to produce murine-human chimeric immunoglobulin-encoding genes. Humanized antibodies can be made using a second approach, i.e., to construct a reshaped human antibody, which has been described in, e.g., Maeda et al., Hum. Antibod. Hybridomas 2: 124-134, 1991, and Padlan, Mol. Immunol. 28: 489-498, 1991.

Unlike the preparation of polyclonal sera, the choice of animal for monoclonal antibody preparation is dependent on the availability of appropriate immortal cell lines capable of fusing with the antibody-producing lymphocytes derived from the immunized animal. Mouse and rat have been the animals of choice for hybridoma technology and are preferably used. For the purpose of making the monoclonal antibodies of the present invention, the animal of choice may be injected with from about 0.01 mg to about 20 mg of the purified MrpH antigen. Typically, the antigen is emulsified in an adjuvant to stimulate general immune responses.

Boosting injections are generally also required. The Lymphocytes can be obtained by removing the spleen or lymph nodes of immunized animals in a sterile fashion, and are fused to immortalized cells. A number of immortalized cell lines suitable for fusion have been developed, and the choice of any particular line is directed by any one of a number of criteria such as speed, uniformity of growth characteristics, deficiency of its metabolism for a component of the growth medium, and potential for good fusion frequency. Intraspecies hybrids, particularly between like strains, work better than interspecies fusions. Several cell lines are available, including mutants selected for the loss of ability to create myeloma immunoglobulin including among these are the following mouse myeloma lines: X63-Ag 8.653, MPC₁₁-X45-6TG, P3 NS1/1-Ag4-1, P3-X63-Ag14 (all BALB/C derived), Y3′Agl.2.3 (rat), and U266 (human). X63-Ag8.653 cells are preferred.

Cloning of hybrid cells can be carried out after 20-25 days of cell growth in selected medium. Cloning can be performed by cell limiting dilution in fluid phase or by directly selecting single cells growing in semi-solid agarose. For limiting dilution, cell suspensions are diluted serially to yield a statistical probability of having only one cell per well. For the agarose techniques, hybrids are seeded in a semisolid upper layer, over a lower layer containing feeder cells. The colonies from the upper layer may be picked up and eventually transferred to wells.

Antibody-secreting hybrid cells can be grown in various tissue culture flasks, yielding supernatants with variable concentrations of antibodies. In order to obtain higher concentrations, hybrid cells may be transferred into animals to obtain inflammatory ascites. Antibody-containing ascites can be harvested 8-12 days after intraperitoneal injection. The ascites contain a higher concentration of antibodies but include both monoclonals and immunoglobulins from the inflammatory ascites. Antibody purification may then be achieved by, for example, affinity chromatography.

The present invention is also directed to the detection of Proteus mirabilis infection by immunological techniques using antibodies directed against MrpH polypeptides. A method is provided for diagnosing Proteus mirabilis infection by contacting a blood or serum sample of an individual to be tested with an antibody directed against an MrpH polypeptide, or an antigenic fragment thereof, for a time and under conditions sufficient to form an antigen-antibody complex and detecting a resultant antigen-antibody complex.

The presence of MrpH polypeptides in a vertebrate blood sample, and therefore the bacteria, can be detected utilizing antibodies prepared as above, either monoclonal or polyclonal, in virtually any type of immunoassay. A method of detecting Proteus mirabilis includes contacting a test sample, such as human serum or human urine, with an antibody directed against an MrpH polypeptide, or an antigenic peptide thereof, for a time and under conditions sufficient to form an antigen-antibody complex. A wide range of immunoassay techniques are available as can be seen by reference to Harlow, et al. (Antibodies: A Laboratory Manual, Cold Spring Harbor Press, (1988)). This, of course, includes both single-side and two-site, or a “sandwich” of the non-competitive types, as well as in traditional competitive binding assays. Sandwich assays are among the most useful and commonly used assays. A number of variations of the sandwich assay technique exist, and all are among the most useful and commonly sued assays. A number of variations of the sandwich assay technique exist, and all are intended to be encompassed by the present invention. Briefly, in a typical forward assay, an unlabeled antibody is immobilized in a solid substrate and a sample of to be tested brought into contact with the bound molecule. After a suitable period of incubation, for a period of time sufficient to allow formation of an antibody-antigen binary complex, a second antibody, labeled with a reporter molecule capable of producing a detectable signal is then added and incubated, allowing time sufficient for the formation of a ternary complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of antigen. Variations on the forward assay include a simultaneous assay, in which both the sample to be tested and the reporter conjugated antibody are first combined, incubated and then added to the unlabeled surface bound antibody. These techniques are well known to those skilled in the art, and the possibly of minor variations will be readily apparent. As used herein, “sandwich assay” is intended to encompass all variations on the basic two-site technique.

MrpH polypeptides may also be detected by a competitive binding assay in which a limiting amount of antibody specific for the MrpH polypeptides is combined with specified volumes of samples containing an unknown amount of the MrpH polypeptides and a a solution containing a known amount of the detectably labeled, e.g., radio-labeled, MrpH polypeptides. Labeled and unlabeled molecules then compete for the available binding sites on the antibody. Phase separation of the free and antibody-bound molecules allows measurement of the amount of label present in each phase, thus indicating the amount of antigen or hapten in the sample being tested. Numerous variations on this general competitive binding assay are known to the skilled artisan and are contemplated by the present invention.

In any of the known immunoassays, for practical purposes, one of the antibodies or the antigen will be typically bound to a solid phase and a second molecule, either the second antibody in a sandwich assay, or, in a competitive assay, the known amount of antigen, may bear a detectable label or reporter molecule in order to allow visual detection of an antibody-antigen reaction. When two antibodies are employed, as in the sandwich assay, it is only necessary that one of the antibodies be specific for the MrpH polypeptide or its antigenic components. The following description provides the methodology for a typical forward sandwich assay; however, the general techniques are to be understood as being applicable to any of the contemplated immunoassays.

In the typical forward sandwich assay, a first antibody having specificity for the mrpH polypeptide or its antigenic components is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking, covalently binding or physically adsorbing the molecule to the insoluble carrier. Following binding, the polymer-antibody complex is washed in preparation for the test sample. An aliquot of the sample to be tested is then added to the solid phase complex and incubated at a 25° C. (or other suitable temperature) for a period of time sufficient to allow binding of any subunit present in the antibody. The incubation period will vary but will generally be in the range of about 2-40 minutes to several hours. Following the incubation period, the antibody subunit solid phase is washed and dried and incubated with a second antibody also specific for the MrpH protein or an antigenic region thereof. The second antibody is linked to a reporter molecule which is used to indicate the binding of the second antibody to the hapten.

By “reporter molecule”, as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphates, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine, 5-aminosalicyclic acid, or tolidine are commonly used. It is also possible to employ fluorogenic substrates, which yield a fluorescent product rather than the chromogenic substrates noted above. In all cases, the enzyme-labeled antibody is added to the first antibody hapten complex, allowed to bind, and then the excess reagent is washed away. A solution containing the appropriate substrate is then added to the ternary complex of antibody-antigen-antibody. The substrate will react with the enzyme linked to the second antibody, giving a qualitative visual signal, which may be further quantitated, usually spectrophotometrically, to give an indication of the amount of hapten which was present in the sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic color visually detectable with a light microscope. As above, the fluorescent labeled antibody is allowed to bind to the first antibody-antigen complex. After washing off the unbound reagent, the remaining ternary complex is then exposed to the light of the appropriate wavelength, the fluorescence observed indicates the presence of the hapten of interest. Immunofluorescence techniques are very well established in the art. However, other reporter molecules, such as radioisotopes, chemiluminescent or bioluminescent molecules, may also be employed. It will be readily apparent to the skilled technician how to vary the procedure to suit the required purpose.

In another embodiment, the antibodies directed against MrpH polypeptides are incorporated into a kit for the detection of P. mirabilis infection. Such a kit may encompass any of the detection systems contemplated and described herein, and may employ either polyclonal or monoclonal antibodies directed against MrpH polypeptides or antigenic regions thereof. Both antibodies complexed to a solid surface described above or soluble antibodies are contemplated for use in a detection kit. The kit can be compartmentalized and includes at least one container containing primary anti-MrpH antibodies and another container containing secondary antibodies covalently bond to a reporter molecule, such that the secondary antibodies are capable of detecting the first antibodies or are themselves directed against MrpH. For example, one contemplated kit is compartmentalized and has the following components: the first container contains killed killed whole cell P. mirabils or MrpH polypeptides as a solution, or bound to a solid surface, to act as a standard or positive control. The second container contains anti-MrpH primary antibodies either free in solution or bound to a solid surface, a third container contains a solution of secondary antibodies covalently bound to a reporter molecule which are reactive against either the primary antibodies or against either the primary antibodies or against a portion of a MrpH polypeptide not reacting with the primary antibody. A fourth and fifth container contains a substrate, or reagent, appropriate for visualization of the reporter molecule.

The subject invention therefore encompasses anti-MrpH antibodies. MrpH antibodies are useful for purification of the MrpH protein and fragments thereof as well as for the detection and study of the Proteus mirabilis bacteria.

Another embodiment of the present invention provides pharmaceutical compositions of the Proteus mirabilis adhesin protein or portions thereof.

Another embodiment of the present invention provides pharmaceutical compositions of the anti-MrpH antibodies.

One embodiment of the present invention provides the purified MrpH protein or protein fragments thereof as a vaccine against MrpH. The vaccine includes an immunogenic amount of an MrpH polypeptide or an antigenic fragment thereof and a pharmaceutically acceptable carrier commonly used in vaccinations. The effective dosage is about 0.5 μg to about 2000 mg of antigen per kilogram of body weight. Boosting regimens may be required and the dosage regimen can be adjusted to provide optimal immunization. Vaccinations can be administered parenterally or extra-parenterally to the mucosal surfaces of the body. The intra-nasal route of administration is preferred.

Another embodiment of the present invention contemplates the treatment of prevention of infection by passive immunization with MrpH antibodies. Pharmaceutical compositions for passive immunization include an amount of anti-MrpH antibodies or antiserum effective in the treatment of Proteus mirabilis infection. The dosage of MrpH anti-serum may be from about 0.01 microliters to about 0.1 milliliters per kilogram of body weight. The dosage of anti-MrpH antibodies depends upon the efficacy and titer of the antibodies but may be from about 0.5 μg to about 2000 mg antibody protein per kilogram of body weight. The dosage regimen can be adjusted to provide the optimum therapeutic response. The active compounds for vaccination or passive immunization may be administered in a convenient manner such as by intranasal, intraperitoneal, subcutaneous, oral, transurethral, intraveneous (where water soluble), intramuscular, or intradermal routes. Intranasal administration is a preferred method of administration but other methods are also contemplated by the present invention. In order to administer protein or anti-MrpH antibodies by other than parenteral administration, the protein or anti-MrpH antibodies should be coated, or administered with a material to prevent inactivation. For example, adhesin protein or anti-MrpH antibodies may be administered in an adjuvant, co-administered with enzyme inhibitors or administered in liposomes. Adjuvants contemplated herein include resorcinols, non-ionic surfactants such as polyoxyethylene oleyl ether and n-hexadecyl polyethylene ether.

The active compounds may also be administered parenterally or intraperitoneally. Dispersions can also be prepared in glycerol, liquid polyethylene glycols, and mixtures thereof, and in oils. Under ordinary conditions of storage and use, these preparations contain a preservative to prevent the growth of undesirable microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extend that easy syringability exists. It must be stable under the conditions of manufacture and storage and must be preserved against the contaminating action of undesirable microorganisms such as fungi. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants.

The prevention of the action of microorganisms can be brought about by various antifungal agents, for example, sorbic acid, thimerosal, and the like. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from previously sterile-filtered solution thereof.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the active material calculated to produce the desired therapeutic effect in association with the required pharmaceutical carrier. The specification for the novel dosage unit forms of the invention are dictated by and directly depend on (a) the unique characteristics of the active material and the particular therapeutic effect to be achieved, and (b) the limitations inherent in the art of compounding such as active material for the treatment of disease.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 μg. Expressed in proportions, the active compound -is generally present in from about 10 μg to about 2000 mg/ml of carrier. In the case of compositions containing supplementary active ingredients, the dosages are determined by reference to the usual dose and manner of administration of the said ingredients.

As used herein “pharmaceutically acceptable carrier” includes any and all solvents, dispersion media, coatings, and antifungal agents, isotonic and adsorption delaying agents, and the like. The use of such media gents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

The MrpH polypeptides may also be optionally conjugated or covalently linked with cholera toxin, preferably non-toxic or inactive cholera toxin or inactivated cholera toxin mutants. For example, MrpH is chemically coupled MrpH to cholera toxin. MalE-MrpH (5 ml of 1 mg/ml) and cholera toxin (5 ml of 2 mg/ml) are covalently linked using the heterobifunctional reagent SPDP (N-succinimidyl 3-(2-pyridyl dithio)propionate; Amersham Pharmacia Biotech) according to the instructions of the manufacturer. For example, for active immunization, the patient is intranasally immunized with MrpH-cholera toxin conjugate (optimal ratio of 20μg MrpH::5 μg cholera toxin) according to the schedule in Table 3. As a negative control, MrpD-cholera toxin conjugate will be used (MrpD is the periplasmic chaperone protein used for assembly of the MR/P fimbriae but not incorporated into the fimbriae or expressed on the surface of the bacterium). As an alternative, the present invention contemplates coadministration of the antigen with unmodified cholera toxin, non-toxic site directed mutants of cholera toxin, or heat labile toxin, for example.

Hence, the above pharmaceutical compositions provide therapeutically administrable forms of an anti-P. mirabilis vaccine, and anti-MrpH antibodies. The vaccine is useful for long-term prevention of P. mirabilis infection and related diseases or syndormes such as, for example, urolithiasis, acute pyelonephritis, and bacteremia as well as preventing the development of bladder and kidney stones in humans. The vaccine is also useful in patients with known anatomically or functionally abnormal urinary tracts, including neurogenic bladders and urinary diversions, patients early in the course of long-term catheterization (urethral, suprapubic, intermittent, and condom), women with apparently normal urinary tracts but who are experiencing recurrent E. coli UTIs (before they develop P. mirabilis infection), immunocompromised individuals and those on immunosuppression drugs, while the anti-sera and antibodies may be used for short-term prevention, including for administration to individuals unable to produce MrpH antibodies and treatment of P. mirabilis infection.

The following non-limiting Examples further illustrate the present invention.

### EXAMPLE 1

Bacterial Strains, Plasmids, and Growth Media

P. mirabilis HI4320 (urease-positive; produced MR/P, Pmf, and ATF fimbriae; hemolysin-positive; tetracycline-resistant) was isolated from a woman with urinary catheter-associated bacteriuria. E. coli strain DH5α λpir was used to maintain the tagged pUT/Mini-Tn5 plasmid pool and E. coli S17 λpir was used as a donor strain for conjugation with P. mirabilis HI4320 (recipient strain; Tet^{r}). E. coli DH5α [supE44 ΔlacU169(φ801acZΔM15) hsdR17 recA1 enda1 gyrA96 thi-1 relal] was used in cloning DNA. Luria broth (per liter, 10 g tryptone, 5 g yeast extract, and 5 g NaCl) and L agar (Luria broth containing 1.5% agar) were used as culture media. Non-swarming agar (per liter, 10 g tryptone, 5 g yeast extract, 5 ml glycerol, 0.4 g NaCl, and 29 g agar) was used to prevent swarming of P. mirabilis (7). Minimal salts medium for Proteus sp. contained, per liter, 10.5 g K₂HPO₄, 4.5 g KH₂PO₄, 0.47 g sodium citrate, and 1.0 g (NH₄)₂SO₄; after autoclave sterilization, 1 ml of 1 M MgSO₄, 10 ml of 20% (V/V) glycerol, and 1 ml of 1% (W/V) nicotinic acid were added to 1 liter of medium prior to pouring. Nitrogen-limited medium (31) consisted of ammonium-free M9 medium (containing, per liter, 6g of Na₂HPO₄, 3g of KH₂PO₄, 0.5g of NaCl, 1 mM MgSO₄, 0.4% glucose, pH7.4) and 10 mM freshly prepared filter-sterilized L-glutamine or L-arginine. Nitrogen-excessive medium contained ammonium-free M9 medium and NH₄Cl (40, 400, and 800 mM). E. coli DH5α (Bethesda Research Laboratories, Gaithersberg, Md.) was used as the host strain for transformation of plasmids other than suicide vector pCVD442 and its derivatives. E. coli DH5α was used for the cloning with 5 the pRK2-derived suicide vector pCVD442.

Molecular Cloning and Nucleotide Sequencing

Chromosomal DNA was isolated from transposon mutants and digested with several restriction enzymes, including EcoRI, SmaI, PvuII, NsiI, DraI, and SspI. Digests were electrophoresed through agarose gels and subjected to Southern blot analysis (as described above) using the kanamycin-resistance gene of pUT/mini-Tn5-Kan (1.7-kb EcoRI-XbaI fragment as a probe. Restriction enzymes which gave rise to hybridizing fragments in the range of 2.5-10 kb were selected for digestion of DNA for cloning. Digested fragments were ligated into appropriately digested pBlueScript (KS-) and transformed into E. coli DH5α. Transformants were selected on LB plates containing kanamycin (50 μg/ml). Sequencing was performed by the dideoxy chain termination method with double-stranded DNA as the template. Reagents from the Prism Ready Reaction Dye Deoxy Termination Kit. (Applied Biosystems) were used in conjunction with Taq polymerase (Boehringer Mannheim Corporation). Reactions were run on a model 373 DNA sequencer (Applied Biosystems).

Expression of MR/P Fimbria in E. coli DH5α and Construction of a DmrpH Mutant

The 9.2-kb AflIII-PstI fragment of mrp gene cluster, including the phase variable mrp promoter fixed in the ON position, structural genes mrpA-H, and a regulatory gene mrpj, was cloned into the AflIII-PstI site of pBluescript. This construct, designated pXL4206, was digested with PvuII to release a 1.2-kb fragment containing the majority of 3′ end of mrpJ and its downstream sequences, and then self-ligated to yield pXL9301. The deletion of mrpJ did not affect the production of normal MR/P fimbriae and therefore the construct pXL9301 and pXL4206 are both referred to as the wild type for MR/P fimbrial production. The construct pXL4206 was digested with EcoRI to release a 1.8-kb fragment that contained the majority of mrpH and its downstream sequences, and then self-religated to form pXL4401 (referred to as DmrpH). To complement this mutant, an 888-bp fragment containing the complete mrpH and its ribosomal binding site was PCR-amplified and cloned into the EcoRV site of pBluescript, resulting in the construct pXL5802. The ON version of mrp promoter (promoter driving transcription of mrpA) was cut from pMRP-ON by AflIII-EcoRI digestion and cloned into NcoI-EcoRI-digested pACYC184, generating the construct pON-184. E. coli DH5α containing both pXL4401 (DmrpH) and pON-184 (vector) was referred to as DmrpH+vector. To place it under the control of the mrp promoter, mrpH was cut out of pXL5802 by SstI-HincII digestion and ligated with SstI-XmnI digested pON-184, resulting in the construct pXL8906. E. coli DH5α containing both pXL4401 (DmrpH) and pXL8906 (mrpH) was referred to as DmrpH+mrpH (see Table 1).

TABLE 1
Summary of mutagenesis studies in E. coli DH5a

| | | Fimbrial ^{a} | | |
|---|---|---|---|---|
| Construct | mrpH | Production | MRHA ^{b} | BA ^{c} |
| pXL4206 or | wild type | + | +++ | +++ |
| pXL9301 | | | | |
| pXL4401 | deletion | − | − | − |
| | (DmrpH) | | | |
| pXL4401 & | wild type | + | +++ | +++ |
| pXL8906 ^{d} | | | | |
| pLX1005 | C66S | + | + | + |
| pLX1105 | C128S | + | − | + |
| pLX1203 | C66S & C128S | + | − | + |
| pLX1305 | C66 & C128 | + | +++ | +++ |

^{a} +, normal fimbrial production; −, less than 1% of normal fimbrial production.
^{b} +++, positive reactions in the first three or more bacterial dilutions; +, only positive in the first dilution; −, negative from the first dilution.
^{c} +++, strong pellicle formation on the broth surface, which breaks into visible bacterial aggregates upon mild vortex; +, weak pellicle formation on the broth surface, which disperses into the broth upon mild vortex; −, no pellicle formation or any kind of bacterial aggregation.
^{d} E. coli DH5a (pXL8906) is negative for fimbrial production, MRHA, and BA.

Amino Acid Residue Substitution (C66S and C128S) of MrpH

Site-directed mutagenesis using overlap extension PCR (Ho, S. N., et al. (1989) Gene 77:51-59, incorporated herein by reference) was applied to amplify mrpH and substitute the 66^{th} codon TGT (encoding cysteine) or the 128^{th} codon TGC (encoding cysteine) with codon TCT (encoding serine). The PCR-amplified mutant mrpH fragments were cloned into pCR®-Blunt (Invitrogen Corp., San Diego, Calif.) and sequenced to confirm the mutations (data not shown). The SnaBI- and KpnI-digested mutant mrpH fragment was used to replace the wild type SnaBI-KpnI fragment of pXL9301 to generate either pLX1005 (C66S) or pLX1105 (C128S). The 1.9-kb SmaI-EcoRI fragment (containing the 66^{th} but not the 128^{th} codon) of pLX1105 was substituted with that of pLX1005 to generate pLX1203 (C66S & C128S), and vice versa, the 1.9-kb SmaI-EcoRI fragment of pLX1005 was substituted with that of pLX1105 to reconstitute the wild type version, designated pLX1305 (referred to as C66 & C128 to be distinguished from the original wild type).

Mannose-resistant Hemagglutination Assay

Bacteria were collected by centrifugation (12,000×g, 25° C., 1 min) after being grown under conditions optimal for MR/P fimbriae production; for P. mirabilis strains, bacteria were passaged statically three times for 48 hrs each passage in Luria Bertani broth at 37° C.; for E. coli strains, bacteria were grown statically at 37° C. for 72 hrs. Cell pellets were suspended in phosphate-buffered saline (PBS) to approximately 10⁹ CFU/ml. A series of two-fold dilutions of bacterial suspension was mixed with equal volume of 3% (v/v) chicken erythrocytes (suspended in 0.85% saline containing 50 mM mannose) in a round-bottomed 96-well microtiter plate. The plate was incubated at room temperature for 30 min to allow erythrocytes to settle to the bottom of the well. Non-agglutinated erythrocytes formed a tight button whereas the agglutinated erythrocytes formed a diffuse mat.

Isolation of MR/P Fimbriae

Bacteria grown under conditions optimal for MR/P fimbrial production, as described above, were collected by centrifugation, resuspended in 10 mM Tris-HCl, pH7.2. MR/P fimbriae were sheared from the cell surface by blending and purified by differential centrifugation as described previously (Li, X. et al. (1998) Infect. Immunity 65:1327-1334, incorporated herein by reference). Partially purified fimbrial preparations were obtained by following the procedure for fimbrial isolation excluding the CsCl gradient centrifugation step.

Expression and Purification of MBP Fusion Proteins

A DNA fragment encoding the mature MrpH (lacking its N-terminal 22-amino acid residue putative signal peptide) was PCR-amplified and cloned into the EcoRI and HindIII sites of pMAL™-C2 (New England Biolabs Inc., Beverly, Mass.) to express MrpH as a C-terminal fusion to maltose binding protein (MBP). As well, a DNA fragment encoding the mature MrpA (lacking its N-terminal 23-amino acid residue putative signal peptide) was PCR-amplified and cloned into the EcoRI and HindIII sites of pMAL™-C2 to express MrpA as a C-terminal fusion to MBP. Expression and purification of MBP-MrpH and MBP-MrpA fusion proteins were carried out as described by Ausubel et al. ((1995) Current Protocols in molecular biology, vol. 2, p.16.6.1-16.6.14. John Wiley & Sons, New York, N.Y., incorporated herein by reference).

Preparation of Antisera against MrpH

The purified MBP-MrpH (100 mg) was emulsified in Freund's complete adjuvant and subcutaneously injected into separate New Zealand White rabbits. Four weeks after the primary immunization, animals were given booster injections of 100 mg protein emulsified in Freund's incomplete adjuvant. Blood samples taken at the 6^{th} week were assayed for reaction with antigen by Western blotting. A second booster injection of 100 mg protein emulsified in Freund's incomplete adjuvant was given to each rabbit at the 7^{th} week. Sera were collected two weeks after the second booster.

The IPTG-induced cell lysate (about 10 mg protein) of E. coli DH5α transformed with plasmid PMALT-C2 was coupled to an AminoLink Plus column (Pierce Inc., Rockford, Ill.) according to the instructions of the manufacturer. Polyclonal antisera from rabbits were passed through the column to remove antibodies against MBP as well as the antibodies reacting with E. coli DH5α proteins. Antibodies bound to the column were eluted through a cycle of pH change as described by Harlow and Lane ((1988) Antibodies: A Laboratory Manual, p. 313-315 Cold Spring Harbor Laboratory, Cold Spring Harbor, N.Y., incorporated herein by reference). The procedure was repeated several times until antisera did not react on a Western blot with the induced cell lysate of E. coli DH5α (pMAL™-C2).

Western Blot Analysis

Partially purified fimbrial preparations were denatured in SDS-gel sample buffer (100° C., 30 min), electrophoresed on a sodium dodecyl sulfate (SDS)-12.5% polyacrylamide gel, and transferred to a polyvinylidene difluoride membrane (Immobilon-P, Millipore corp., Bedford, Mass.). The blot was incubated with rabbit polyclonal antiserum against MrpH, followed by incubation with goat anti-rabbit IgG alkaline phosphatase conjugate, and then developed with BCIP/NBT (5-bromo-4-chloro-3-indolylphosphate toluidinium/nitro blue tetazolium) as a chromagenic substrate for alkaline phosphatase.

Immunogold Electron Microscopy

Immunogold labeling was performed by a modification of the method of Faulk and Taylor ((1971) Immunochemistry 8:1081-1083, incorporated herein by reference). Bacteria were grown under conditions optimal for MR/P fimbrial production. A drop of bacterial culture was placed on a Formvar-coated grid and processed as described previously (Li, X. et al. (1997) Infect. Immun. 65:1327-1334, incorporated herein by reference). The grid was incubated at 37° C. for 30 min each time with 1:100 dilution of rabbit antiserum against MrpH followed by 1:25 dilution of goat anti-rabbit IgG (H+L) conjugated with 30-nm-diameter gold beads (AuroProbe™ EM GAR G30, Amersham Life Science, Amersham Place, England), and then with 1:100 dilution of rabbit antiserum against MR/P fimbria (Li, X. et al. (1997) supra) or rabbit antiserum against MrpA followed by 1:25 dilution protein G conjugated with 5-nm-diameter gold beads (AuroProbe EM protein GG5; Amersham Life Science). Between each incubation, grids were washed three times with PBS containing 1% bovine serum albumin (BSA). At the end, grids were washed three times with PBS containing 1% BSA and three times with distilled water, negatively stained with 1% sodium phosphotungstic acid (pH6.8), and examined by transmission electron microscopy with a JEM-1200EX II electron microscope (JEOL Ltd., Tokyo, Japan).

CBA Mouse Model of Ascending Urinary Tract Infection

CBA mice were transurethrally challenged with 10⁶ to 10⁷ CFU bacteria per mouse using the method described previously (Li, X. et al. (1997), supra). After 7 days, mice were sacrificed and bacteria recovered from urine, bladder, and kidneys were enumerated on nonswarming agar plates with appropriate antibiotics. The range of detection in this assay is 10² to 10⁹ CFU/ml urine or CFU/g tissue. Values ≦10² were set to 102, and values ≧109 were set to 10⁹.

Signature-tagged Mutagenesis

Plasmid DNA representing the tagged pool [pUT/miniTn5 marked with random signature-tags (Ampr) (26)] was transformed into donor strain E. coli S17 λpir by electroporation. Transformants were pooled and stored in Luria broth with 15% (W/V) glycerol at −70° C. Recipient cells of P. mirabilis HI4320 (TetR) were grown overnight at 37° C. in Luria broth containing tetracycline (20 μg/ml). Overnight culture (50 μl) was inoculated into 5 ml fresh Luria broth and grown to an OD₆₀₀=0.5-1.0; a large loop-full of frozen donor cells S17 λpir harboring the tagged pUT/Mini-Tn5 plasmid was inoculated into 3.5 ml of Luria broth and grown for 1.5 hrs to allow recovery of bacteria. In a 96-well microtiter plate, 50 μl of each donor and recipient cells was mixed and a 48-well metal replicator was used to transfer samples of the strain suspensions from the microtiter dishes onto the non-swarming Luria agar plate. After overnight mating and growth, each mating suspension was restreaked onto non-swarming Luria agar plates containing kanamycin (50 μg/ml) and tetracycline (20 μg/ml). A single colony from each mating was selected at random and stored in a single microtiter plate well.

Southern Blot Analysis

Chromosomal DNA was digested with either PvuII or PvuII and PvuI, electrophoresed on a 0.8% agarose gel, and transferred to a membrane (QIABRANE Nylon plus; Qiagen Inc., Chatsworth, Calif.). Probe labeling, hybridization, and signal detection were carried out with the ECL direct nucleic acid labeling and detection system (Amersham Life Science, Amersham Place, England) according to the instructions of the manufacturer.

Construction of an Isogenic mrpH::aphA Mutant of HI4320

A 1.8-kb StyI-PvuII fragment of mrp gene cluster that contains part of mrpG, mrpH, and part of mrpJ was cloned into pBluescript (Stratagene, La Jolla, Calif.). A kanamycin-resistance (encoded by aphA) cassette (the 1.3-kb BamHI fragment of pUC4k from Pharmacia Biotech Inc., Piscataway, N.J.) was inserted into the BamHI site within mrpH, so that it was flanked with approximately 1.0 kb mrp sequence on its 5′ side and 0.8 kb on its 3′ side. This disrupted mrpH, along with its flanking homologous sequence, was cloned into pCVD442, a pRK2-derived suicide vector, and electroporated into HI4320. About 500 kanamycin-resistant transformants were obtained on Luria-Bertani agar plates containing kanamycin (50 mg/ml). Of these, 400 were picked and passaged on to Luria-Bertani agar plates containing ampicillin (100 mg/ml) to screen for ampicillin-resistance. Nineteen of 400 transformants were ampicillin-susceptible and considered as possible mrpH::aphA mutants. Four of the nineteen ampicillin-susceptible, kanamycin-resistant transformants (mrpH1011, mrpH1022, mrpH1023, and mrpH1100) were confirmed by Southern analysis as mrpH::aphA mutants.

### EXAMPLE 2

Insertional Mutagenesis Studies in P. mirabilis

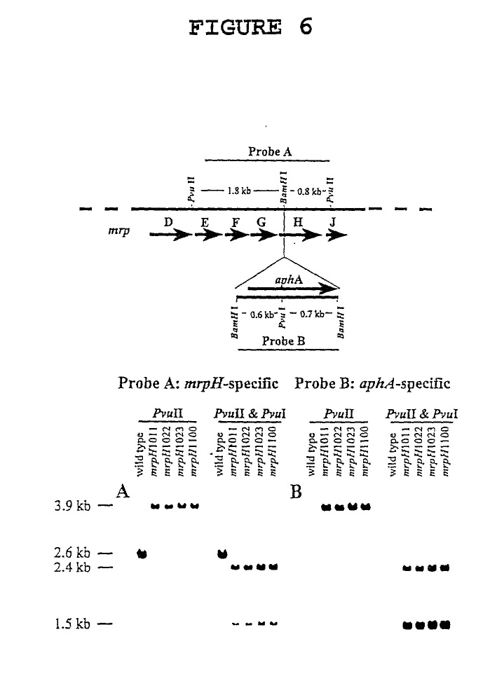

Insertional mutagenesis was carried out to disrupt mrpH in the clinical isolate of P. mirabilis, strain HI4320, from which the mrp gene cluster was originally isolated. A kanamycin-resistance (encoded by aphA) cassette was introduced into the BamHI site within mrpH on the chromosome through allelic exchanges (see Materials and Methods). The occurrence of the insertional mutation was verified by Southern blotting (FIG. 6). When probed with the mrpH-specific sequence (Blot A), the 2.6-kb PvuII fragment reacted in the wild type strain but was shifted to 3.9-kb in the mrpH::aphA mutants, suggestive of a 1.3-kb insertion (size of the aphA insert). When probed with the aphA-specific sequence (Blot B), the 3.9-kb band in the mutants was hybridized with the probe, suggesting that the 1.3-kb insertion represents the kanamycin-resistance cassette. The mutation was further confirmed by PvuII and PvuI double digestion. Since the kanamycin-resistance cassette insertion also introduced a PvuI site into this region, PvuI cleaved the 3.9 kb fragment of the mrpH::apIhA mutants into two fragments with predicted size (2.4 kb and 1.5 kb). It was concluded from the Southern blotting results that the mrpH was disrupted by a kanamycin-resistance cassette in these mrpH::aphA mutants.

Immunogold eletron micrography (EM) studies showed that the mrpH::aphA mutants were not capable of producing a normal complement of MR/P fimbriae. Indeed, less than 1% of the bacteria were MR/P fimbriated in the mrpH::aphA mutants versus about 50% in the wild type. On the few mutant bacteria that did produce MR/P fimbriae, there was much fewer of these fimbriae per bacterium compared to the wild type strain and fimbriae produced by the mutant strains were often shorter and amorphous (data not shown). However, even though the mrpH::aphA mutants were defective in producing MR/P fimbriae, they were still positive for mannose-resistant hemagglutination (data not shown), suggesting that the MR/P fimbria may not be the only mannose-resistant hemagglutinin produced by the wild type P. mirabilis HI4320. This study confirmed that MrpH was the functional adhesin of MR/P fimbria.

### EXAMPLE 3

Protection from Homologous Challenge by P. mirabilis Following Experimental Infection

To assess immunity to reinfection with a homologous strain of P. mirabilis following experimental infection, one group (N=20) of CBA mice was inoculated transurethrally with 5×10⁷ CFU P. mirabilis HI4320. After 4 weeks this group of mice and a control group were treated with ampicillin for 3 days followed by a 4 day washout period. Both groups were then challenged with a Nal^{R} mutant of the homologous strain. One week later, animals were sacrificed and urine, bladder, and kidneys were quantitatively cultured. Mean log₁₀ CFU/ml for the control vs vaccinated groups were: urine, 5.4 vs 3.8; bladder, 5.0 vs 3.3; and kidney, 4.1 vs 2.6 (P=0.17). ELISA using whole cell preparation of P. mirabilis showed high serum antibody titers. To identify antigens to which mice responded, sera from selected mice were used for Western blot analysis of protein preparations from the parent strain and isogenic mutants lacking MR/P fimbriae, PMF fimbriae, urease, hemolysin, and flagella. Strong immunoglobulin responses were identified for MR/P and PMF fimbriae and flagella in addition to numerous other unidentified surface antigens.

Thus, a significant serum Ig response developed in mice with experimental P. mirabilis UTI which correlated with only modest protection against homologous challenge.

### EXAMPLE 4

Vaccination using Whole Killed P. mirabilis and Purified MR/P Fimbriae

To test the effect of using crude and purified antigen preparations, we immunized groups of 10 CBA mice on days 0, 7, and 14 with killed whole cells of P. mirabilis given with 10 μg of cholera toxin or with MR/P fimbriae covalently coupled to cholera toxin. Four routes of immunization were tested: intranasal (IN), transurethral (TU), subcutaneous (SC), and oral (OR).

On day 21 immunized and naive mice were challenged transurethrally with P. mirabilis HI4320. Seven days after challenge urine, bladder, and kidneys were quantitatively cultured.

For whole killed P. mirabilis immunization, significant decreases in colonization were induced by intranasal (urine, p<0.01; bladder, p=0.006; kidneys; p=0.056) and subcutaneous (urine, p=0.002; bladder, p=0.006; kidneys; p=0.07) immunization (see Table 2). After intranasal and subcutaneous immunization, 88% and 60% of mice, respectively had undetectable numbers of bacteria in their kidneys one week after transurethral challenge; this compared to only 10% of unimmunized mice that were protected.

TABLE 2

| | | | No. mice w/ | | | |
|---|---|---|---|---|---|---|
| | | | <10² CFU/g | | | |
| | | | kidney (% | | log₁₀ CFU/g | log₁₀ CFU/g |
| Antigen | N | Route | protected) | | bladder | kidney |
| None (control) | 1 | — | 1/10 | (10) | 6.39 | 4.65 |
| | 0 | | | | | |
| Whole-killed | 8 | IN | 7/8 | (88) | 2.18 | 1.56 |
| P. mirabilis + | 1 | SC | 6/10 | (60) | 2.04 | 1.89 |
| cholera toxin | 0 | | | | | |
| | 9 | TU | 3/9 | (33) | 3.76 | 3.19 |
| | 7 | OR | 2/7 | (29) | 4.23 | 4.19 |

For MR/P fimbriae immunization, significant reduction in colonization was seen with intranasal (urine, p=0.04; bladder, p=0.02; kidneys; p=0.008) and transurethral (urine, p<0.06; bladder, p=0.03; kidneys; p=0.002) immunization (see Table 3). After intranasal and transurethral immunization, 70% and 83% of mice, respectively, had undetectable numbers of bacteria in their kidneys one week after transurethral challenge; this compared to only 10% of unimmunized mice that were protected. IgA and IgG were detectable in vaginal washes only after intranasal immunization.

TABLE 3

| | | | No. mice w/ | | | |
|---|---|---|---|---|---|---|
| | | | <10² CFU/g | | log₁₀ | log₁₀ |
| | | | kidney (% | | CFU/g | CFU/g |
| Antigen | N | Route | protected) | | bladder | kidney |
| None | 1 | — | 1/10 | (10) | 6.39 | 4.65 |
| (control) | 0 | | | | | |
| MR/P | 1 | IN | 7/10 | (70) | 1.84 | 1.37 |
| fimbriae- | 0 | | | | | |
| cholera | 8 | SC | 2/8 | (25) | 3.92 | 4.85 |
| toxin | 6 | TU | 5/6 | (83) | 1.61 | 1.67 |
| | 9 | OR | 4/9 | (44) | 4.50 | 3.71 |

Intranasal immunization with killed whole cells or MR/P fimbriae and a mucosal adjuvant offered significant protection against experimental UTI and elicited vaginal antibody production.

### EXAMPLE 5

Immunization with Purified MrDH, the Adhesin of MR/P Fimbriae

To examine the effect of using the fimbrial adhesin subunit as antigen, a maltose binding protein (MBP) fusion of MrpH (surface-expressed adhesin) was overexpressed and purified on an amylose column. MBP-MrpH was conjugated to cholera toxin (CT) and used to immunize mice either subcutaneously or intranasally (initial and booster doses of ˜20 μg/mouse). Mice were boosted after 1 and 2 weeks. After 3 weeks, mice (groups of 11-12) were challenged transurethrally with ˜1×10⁷ CFU of P. mirabilis HI4320. One week after challenge, mice were sacrificed and urine, bladder, and kidneys were quantitatively cultured. Kidneys of mice intranasally immunized with MBP-MrpH yielded significantly fewer (p=0.002) bacteria (log₁₀ CFU=3.35) than naive mice (log₁₀ CFU=6.10).

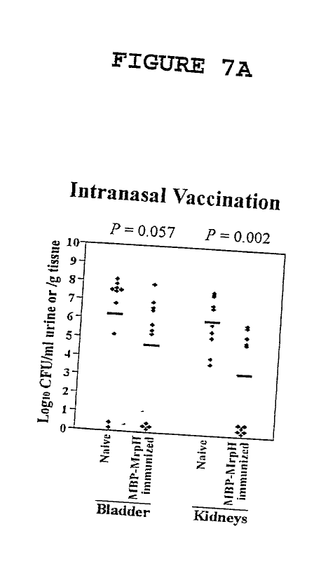

Thus, intranasal immunization with MrpH conjugated to cholera toxin reduced the number of bacteria in the kidneys of immunized mice by >80-fold compared to a naive control (FIGS. 7A, 7B, & 7C).

### EXAMPLE 6

Analysis of Virulence in a Mouse Model of Ascending Urinary Tract Infection

The contribution of MR/P fimbria to the colonization and virulence in the urinary tract infection was assessed in a CBA mouse model of ascending urinary tract infection.

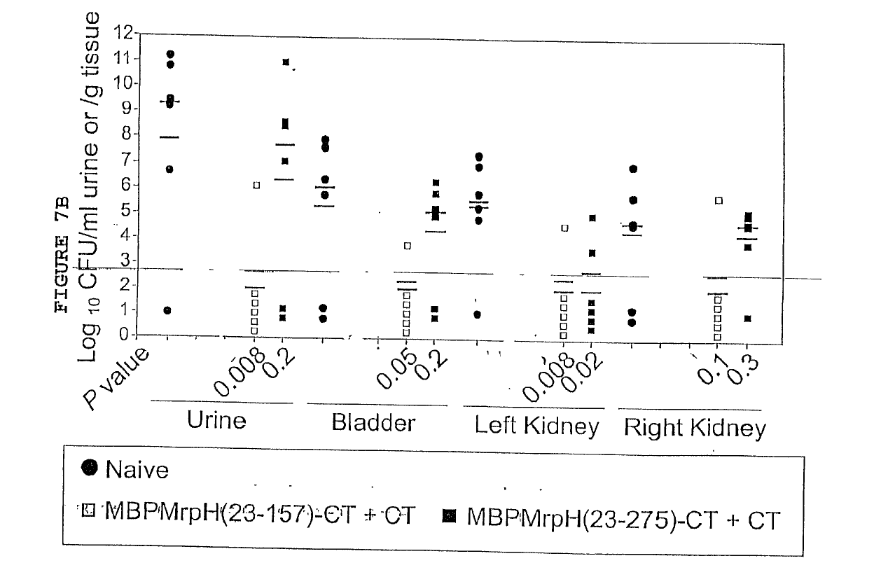

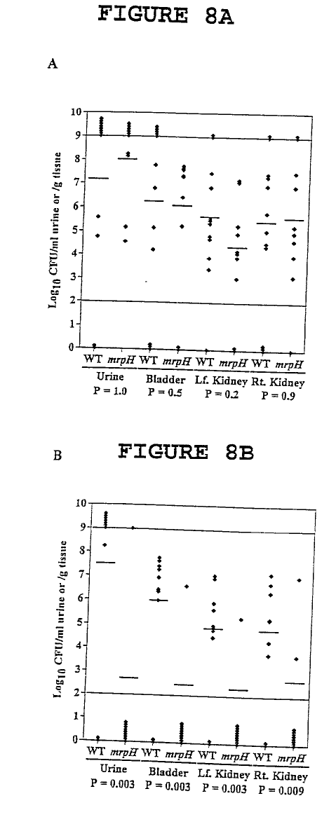

In a challenge experiment, a group of 12 CBA mice was challenged with 4.8×10⁶ CFU/mouse of the wild type HI4320, and another group of 10 CBA mice was challenged with 5.5×10⁶ CFU/mouse of the mrpH::aphA mutant. After 7 days, mice were sacrificed and bacteria recovered from urine, bladder, and kidneys were enumerated on nonswarming plates. Bacteria recovered from mice infected with the mrpH: :aphA mutant were enumarated on both nonswarming plates and nonswarming plates containing kanamycin (50 mg/ml). No detectable kanamycin-sensitive revertants were found, indicating that the mutation is stable at least during the 7-day infection period. The geometric mean values of bacterial count were as follows: urine, 7.19 (wild type) versus 8.02 (mutant) log₁₀ CFU/ml (P=1.0); bladder, 6.25 (wild type) versus 6.09 (mutant) log₁₀ CFU/g (P=0.5); left kidney, 5.63 (wild type) versus 4.38 (mutant) log₁₀ CFU/g (P=0.2); right kidney, 5.46 (wild type) versus 5.64 (mutant) log₁₀ CFU/g (P=0.9) (FIG. 8A). The data showed that the mrpH: aphA mutant colonized the urinary tract of CBA mice just as well as the wild type strain did.

A dramatically different outcome was observed in a cochallenge experiment. A group of 10 CBA mice was challenged with a roughly 1:1 mixture of the wild type HI4320 and its isogenic mrpH: :aphA mutant (7.9×10⁶ CFU of the wild type and 1.2×10⁷ CFU of the mutant were inoculated into the bladder of each mouse). After 7 days, mice were sacrificed and bacteria recovered from urine, bladder, and kidneys were enumerated on both nonswarming plates, where both the wild type HI4320 and the mrpH::aphA mutant would grow, and nonswarming plates containing kanamycin (50 mg/ml), where only the mrpH::aphA mutant would grow. The geometric mean values of bacterial count were as follows: urine, 7.52 (wild type) versus 2.70 (mutant) log₁₀ CFU/ml (P=0.003); bladder, 5.98 (wild type) versus 2.46 (mutant) log₁₀ CFU/g (P=0.003); left kidney, 4.87 (wild type) versus 2.33 (mutant) log₁₀ CFU/g (P=0.003); right kidney, 4.82 (wild type) versus 2.67 (mutant) log₁₀ CFU/g (P=0.009) (FIG. 8B). The mrpH: aphA mutant was shown to be significantly less competitive than the wild type strain in the ability to colonize the lower and upper urinary tract of mouse.

### EXAMPLE 7

Identification of MrpH as the MR/P Hemagglutinin of Uropathogenic P. mirabilis

mrpH was identified downstream of mrpG in the mrp gene cluster encoding MR/P fimbriae of uropathogenic P. mirabilis. Since the predicted MrpH shared 30% amino acid sequence identity with PapG, the Galα(l-4)Gal-binding adhesin of E. coli P fimbriae (most heavily conserved at the C-terminus where it interacts with the chaperone protein), it was believed that mrpH encoded the functional MR/P hemagglutinin. MR/P fimbriae, expressed in E. coli DH5α, conferred on bacteria both the ability to cause mannose-resistant hemagglutination and to aggregate to form a pellicle on the broth surface. Both a DmrpH mutant expressed in E. coli DH5α and an isogenic mrpH::aphA mutant of P. mirabilis were unable to produce normal MR/P fimbriae efficiently (much fewer and shorter fimbriae than wild type), which indicated that MrpH was also involved in fimbrial assembly. In an attempt to separate fimbria-biogenesis from receptor-binding, site-directed mutagenesis was undertaken. Amino acid residue substitution of the N-terminal cysteine residues (C66S, C128S) of MrpH abolished receptor-binding activity (hemagglutinating ability) of MrpH but allowed normal fimbrial assembly, supporting the thesis that MrpH was the functional MR/P hemagglutinin. Immunogold electron microscopy of P. mirabilis HI4320 revealed that MrpH was located at the tip of MR/P fimbriae, consistent with its role in receptor-binding. The isogenic mrpH::aphA mutant of HI4320 was less able to colonize the urine, bladder, and kidneys in a mouse model of ascending urinary tract infection (P<0.01).

While there were similarities between P. mirabilis MR/P and E. coli P fimbriae, there were more notable differences: a) synthesis of the MrpH adhesin was required to initiate fimbriae assembly; b) MR/P fimbriae conferred an aggregation phenotype; c) site-directed mutation of specific residues abolished receptor-binding but allowed fimbrial assembly; and d) mutation of the adhesin gene abolished virulence in a mouse model of ascending urinary tract infection.

### EXAMPLE 8

Studies by Langermann et. al. ((1997) Science 276:607-611, incorporated herein by reference) showed that FimH-adhesin-based systemic vaccination prevented type-l-piliated E. coli colonization in mice. MrpH-MrpD (adhesin-chaperone) complexes were prepared in order to express the MrpH adhesin in its native conformation as was done for FimC-FimH conjugates for an E. coli type 1 fimbrial vaccine Langermann et. al. (1997) supra. To accomplish this, MrpH was copurified with its chaperone protein MrpD which, by analogy to homologs in the E. coli P fimbriae system, capped the protein in the periplasmic space and prevented polymerization until the adhesin protein was translocated across the outer 1 membrane.

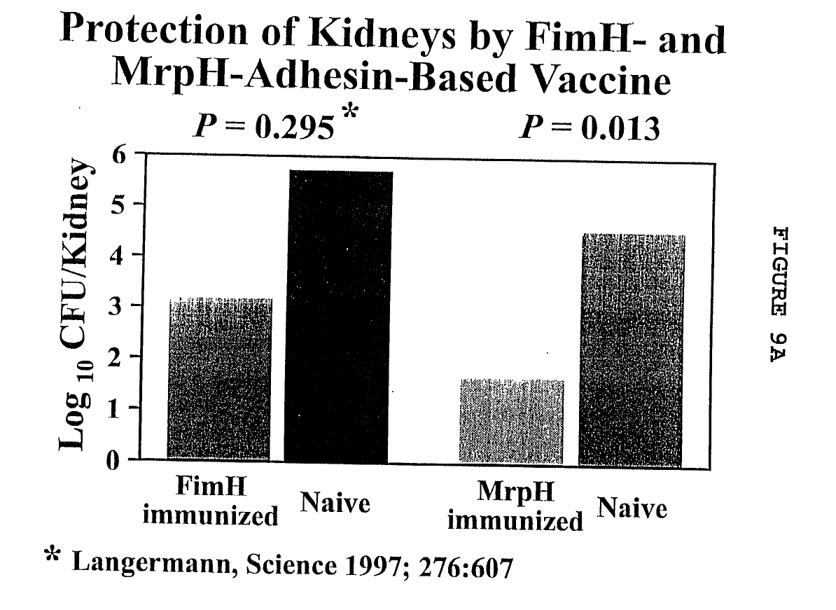

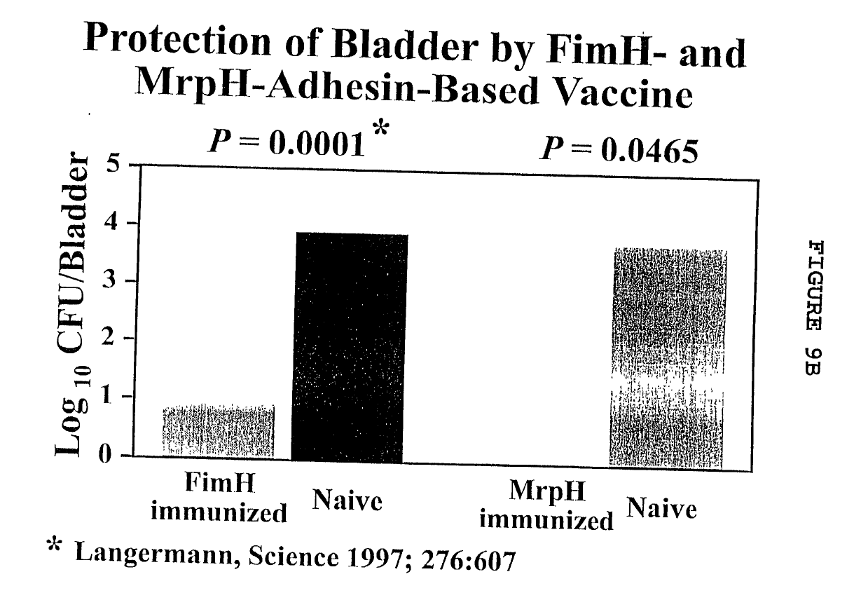

MrpH-MrpD immunization protected bladder and kidneys of mice from P. mirabilis colonization (n=10) to a greater extent than the FimH-based vaccine of Langermann et al. (FIGS. 9A & 9B)

### EXAMPLE 9

mrpH is conserved among strains. A fragment carrying the open reading frame was PCR-amplified from 15 strains using primers corresponding to the 5′ and 3′ end of mrpH (Mobley851: GAAGATTGAGGTTTTACATGTTTAT (SEQ ID NO:12) Mobley824: CAAAATCAAAATTCATCATCATAAT (SEQ ID NO:13). The MrpH genes was amplified from 5 pyelonephritis strains, 5 catheter-associated bacteriuria strains, and 5 fecal strains of P. mirabilis. The amplified fragments were subjected to nucleotide sequencing. Nucleotide sequences and predicted amino acid sequences were aligned using Multiple sequence alignment program (http://dot.imgen.bcm.tmc.edu:9331/multi-align/multi-align.html).

### EXAMPLE 10

Translational fusion of MrpH-CtxB

The toxic A1 domain of cholera toxin was replaced with MrpH, the MR/P fimbrial adhesin. The binding and immunomodulatory properties of the cholera toxin B subunit were maintained. The pBluescript-derived plasmid pLDR19 was used to express the recombinant MrpH-cholera toxin A2 fusion protein and cholera toxin B subunit under the control of the inducible lac promoter. The signal sequence of LTIIb B subunit of type II heat-labile enterotoxin of E . coli was used to direct secretion of the recombinant protein to the periplasm of the host bacterium. The protein, recovered by osmotic shock or lysis, was purified on resins to which the cholera toxin B subunit bound. This purified preparation was used directly for intranasal immunization.

Chemical Coupling of MrpH to Cholera Toxin

MrpH was also chemically coupled to cholera toxin. MalE-MrpH (5 ml of 1 mg/ml) and cholera toxin (5 ml of 2 mg/ml) was covalently linked using the heterobifunctional reagent SPDP (N-succinimidyl 3-(2-pyridyl dithio)propionate; Amersham Pharmacia Biotech) according to the instructions of the manufacturer. As a control, MalE-MrpD was conjugated to cholera toxin.

### EXAMPLE 11

Maltose Binding Protein (MBP) MBP-MrpH Fusions

To facilitate purification of the chaperone and adhesin, separate translational fusions of MalE (maltose binding protein) with MrpH were constructed using vector pMalc2 (New England Biolabs). MBP was fused to the N-terminus of MrpH. Protein expression was induced with IPTG in E. coli DH5α containing the constructs. Induction was verified using SDS-PAGE. Lysates from the induced cultures were passaged through an amylose column and the column was washed. Fusion proteins were eluted from the column with 10 mM maltose and assessed for purity by SDS-PAGE.

MrpD-MrpH (Chaperone-adhesin) Fusion

Separate MalE-MrpD and MalE-MrpH fusions were mixed, concentrated and dialyzed against PBS. The protein complex (MrpD-MrpH) was then conjugated to cholera toxin using the SPDP reagent (see Example 10). These preparations were used for intranasal vaccination.

### EXAMPLE 12

Expression of MrpH in an DKK223-3 Prokaryotic Expression System

The pKK223-3 vector contains the strong trp-lac (tac) promoter first described by deBoer, et al. (1983, Proc. Nat. Acad. Sci., USA). The tac promoter contains the −35 region of the trp promoter and the −10 region, operator, and the ribosome binding site of the lac UV-5 promoter. In a lac i^{Q} host such as E. coli JM 105, the tac promoter is repressed and may be derepressed at the appropriate time by the addition of IPTG. The tac promoter is followed by a polylinker derived from pUC8 (Vieria and Messing, 1982, Gene 17: 259-268) containing unique EcoRI, BamHI, SalI, PstI, SmaI, and HindIII restriction enzyme sites which facilitate the positioning of genes behind the promoter and ribosomal binding site. The polylinker is followed by a DNA segment containing the strong rrnB ribosomal RNA transcription terminators which have been previously characterized (Brosius, et al., 1981, J. Mol. Biol., 148:107; and Brosius, et al., 1981, Plasmid, 6:112). The position of the terminators stabilizes the host-vector system, presumably by inhibiting the over expression of detrimental RNA species or proteins by the strong tac promoter contained on the vector (Gentz, et al., 1981, Proc. Nat. Acad. Sci., USA 78:4936; Brosius, 1981, supra,). The remainder of the plasmid consists of pBR322 sequences.

Expression of MrpH polypeptides within a pKK233-3 vector is induced by introduction of IPTG to the culture medium when using E. coli JM105 as host cells. A MrpH_polypeptide is detected by comparison of the electrophoretic patterns of proteins derived from JM105 cells having the MrpH expression vector, with the pattern from cells that do not. SDS polyacylamide gels stained with an appropriate protein stain, such as Coomassie Blue, may be used for these comparisons, or, Western blot analysis using anti-MrpH antibodies to detect a MrpH polypeptide, may be employed (see for example, Harlow, et al., 1988, Antibodies: A Laboratory Manual, Cold Spring Harbor Laboratory Press).

The MrpH polypeptide(s) expressed in a pKK233-3 expression vector system are purified by preparative SDS gel electrophoresis, or by immunoaffinity chromatography with an anti-MrpH antibody.

### EXAMPLE 13

Expression of MrPH in a Baculovirus Expression System

To obtain regulated expression of an MrpH polypeptide, the coding region of the MrpH gene is placed downstream of a strong baculovirus promoter, the polyhedrin promoter, within a transfer vector. The transfer vector is a plasmid which has been genetically engineered to contain baculovirus DNA flanking the polyhedrin gene, as well as convenient restriction enzyme recognition sites adjacent to the strong polyhedrin promoter. Transfer vectors are cotransfected with baculovirus DNA to allow homologous recombination between the baculovirus DNA within the transfer vector and the genome of the baculovirus. Such homologous recombination replaces the polyhedrin coding region in the baculovirus genome with the MrpH coding region. This replacement caused the polyhedrin gene product to be lost and gives rise to an occlusion negative viral phenotype. Hence, baculoviruses which have incorporated the MrpH coding region are recognized by an occlusion negative phenotype. Techniques for distinguishing this phenotype, as well as for manipulating transfer vectors, and recombinant baculoviruses are provided in Summers et al. (1987) A Manual of Methods for Baculovirus Vectors and Insect Cell Culture Procedure, Texas Agricultural Experiment Station, Bulletin No. 1555.

To place the coding region into a transfer vector, DNA is digested with the appropriate restriction enzymes and the DNA fragment encoding MrpH amino acids 23-275 (or any portion thereof) is isolated. The transfer vector, is digested with a restriction enzyme that cuts just downstream of the polyhedrin promoter. After ligation (T4 DNA ligase, New England Biolabs) of the fragment encoding the MrpH coding region to the linearized transfer vector, recombinants are selected for ampicillin resistance and restriction mapped to identify those having the correct structure.

The recombinant MrpH transfer vector is co-transfected with baculovirus AcMNPV DNA, into S. frugiperda Sf9 cells by the method described in Summers et al. The plaques are screened for an occlusion negative phenotype, and several recombinant (occlusion-negative) baculovirus clones are separately plaque purified three times to ensure that the MrpH recombinant clones are homogeneous.

### EXAMPLE 14

Generation of Polyclonal Antibodies

To prepare polyclonal antibodies directed against MrpH polypeptides, any of a variety of antigens are used, such as a purified MrpH fusion protein, a purified polypeptide or a synthetic peptide encoding a portion of a MrpH polypeptide.

A synthetic peptide with the following amino acid sequence was made for immunization into rabbits:(MrpH 23-157) MASIFSYI TESTGTPSNA TYTYVIERWD PETSGILNPC YGWPVCYVTV NHKHTVNGTG GNPAFQIARI EKLRTLAEVR DWLKNRSFP IEGQTTHRGP SLNSNQECVG LFYQPNSSGI SPRGKLLPGS LCGIAPP

This MrpH peptide was identified as a strongly antigenic epitope.

New Zealand white female rabbits were immunized by subdermal injection with 100 μl of Fruend's complete adjuvant containing 0.1-1 of oligopeptide in multiple locations along the back. The rabbits were first shaved on both sides of the back for easy subdermal injection. Typically rabbits were boosted with similar amounts of antigen at 10-40 days intervals following the primary injection, until the serum was positive for MrpH reactivity at a dilution of greater than 10⁻⁴ when assayed by ELISA, immunoblotting, and immunoprecipitation analysis.

### EXAMPLE 15

Monoclonal Antibody Production

Monoclonal antibodies are prepared in accordance with the techniques developed by Kohler and Mulskin (Eur. J. Immunol. 6:115-519, 1976) and Harlow et al. (Antibodies: A Laboratory Manual, Cold Spring Harbor Laboratory Press, 1988). Balb/c mice are immunized subdermally with 100 μl of Freund's complete adjuvant containing 0.1-1 mg of an MrpH antigen such as a purified MrpH polypeptide or fusion protein, or the conjugated or non-conjugated MrpH peptides described above. Two weeks after the initial injection, the mice are boosted with the appropriate antigen by intravenous and intraperitoneal injection of about 100 ug of antigen in phosphate buffered saline (PBS).

Five days after the last injection and after confirmation of the presence of antibody in mouse sera, the mice are sacrificed and their spleens removed. Spleen cells are obtained by gentle disruption of the spleen in a 7 ml Dounce homogenizer in 3.5-4 ml PBS. The cells are then pelleted at 1200 rpm in a PR6 centrifuge for 6 minutes at room temperature. The supernatant is removed into a suction flash, and the cells are resuspended in 15 ml 0.83% NH₄Cl. The cells are again pelleted by centrifugation for 8 minutes, at 1200 rpm at room temperature, then the supernatant is withdrawn into a suction flask cells resuspended in 20 ml PBS.

The following solutions are prepared for use in the subsequent cell fusion:

Hypoxanthine (H), 680 mg/100 ml H₂O; add 204 drops conc. H₂SO₄; heat to dissolve,

Aminopterin (A), 46.4 mg/100 ml H₂O; add 2 drops 1.0 N NaOH to dissolve,

Thymidine (T), 775 mg/100 ml H₂O; add 45 mg glycine PEG-DME-melt PEG at 42° C., then add 1 ml DME (at 37° C.); adjust pH with 1.ON NaOH to 7.6,

DMEM-to 500 ml DME add 37.5 ml a horse-serum; 37.5 ml FCS, 10.0 ml L-glutamine, 0.2 ml garamycin,

2X HAT-DME-to 200 ml DME add 25.0 ml a-horse serum, 25.0 ml FCS, 4.0 ml L-glutamine, 0.2, garamycin, 0.8 ml H, and 0.8 ml A, and 0.8 mlT (2X HT-DME omits A),

Cloning Agar—350 mg unwashed Difco agar in 25 ml H₂O, autoclaved,

Cloning Medium—to 25 ml 2X DME, add 35 ml filtered, condition DMEM, 7 ml a-horse serum, 7 ml FCS, 1 ml L-glutamine, 0.1 ml garamycin.

Two 30 ml flasks of X63-Ag8.653 myeloma cells are added to centrifuge tubes and spun down at 1200 rpm for 8 minutes at room temperature. The spleen cells are resuspended in 20 ml PBS. For each suspension, 0.01 ml is removed and added to 0.1 ml 0.4% trypan blue and 0.3 ml PBS for cell counting. The volume of each suspension is adjusted so as to obtain a spleen cell to X63-Ag8.653 cell ratio of 10:1, and the suspensions are then mixed. The mixture is pelleted at 1200 rpm for 8 minutes at room temperature and all but about 0.1 ml of supernatant removed. The cells are then resuspended in the remaining liquid and added to 1.3 ml of 1:1 PEG-DME solution, pH 7.6. Every minute the volume of the solution is doubled with DME until the final volume is 25 ml.

The cells are again pelleted, the supernatant decanted, and the cells resuspended in enough 50% 2X HAT-DME/50% condition DMEM (the supernatant retained from the X63-Ag8.653 cells above) to yield a final concentration of about 3.5×10⁶ spleen cells. The cells are distributed into a 96-well flat-bottom microtiter plate (TC-96; Flow Laboratories), at 0.1 ml/well. The plate is incubated at 37° C. in humidified air/CO₂ until visible colonies appear, usually about 10-12 days. The contents of the well is transferred to 0.5 ml of HAT-DME/conditioned DME in a TC-24 plate (Flow Laboratories). When healthy cell growth appears (about 2-5 days), about 0.35 ml medium is removed and tested for antibody production by enzyme-linked immunosorbent assay (ELISA), immunoprecipitation of MrpH polypeptides, or Western blot analysis. When cells producing the antibodies of interest are growing well, one drop of each culture is transferred into 1.0 ml DMEM in a TC-24.

To clone the hybrid cells, 25 ml of melted agar and 76 ml of cloning medium is combined, and 5 ml is pipetted into 60 mm petri dish and left to solidify. Cells from DMEM cultures are diluted in 50% DMEM/50% conditioned DMEM, at 10⁻¹ or 10⁻² dilutions depending on cell growth. Into sterile tubes is placed 0.1 ml of each of the two dilutions, and to each is added 0.9 ml of cloning medium/agar mixture. This is mixed well and poured over the surface of the agar underlay. After solidification the plates are incubated at 37° C. in a CO₂ incubator until colonies are visible with the naked eye, typically about 7-10 days. Colonies are then picked and transferred 0.1 ml of DMEM/conditioned DMEM in a TC-99 plate and incubated at 37° C. in a CO₂ incubator. After the culture is acidic (usually 1-4 days), transfer is made to 0.05 ml DMEM in TC-24 plate. When the growth is 50% confluent, the medium is removed and tested for antibody production as previously. Those clones producing specific antibodies are moved into 5 ml DMEM in 25 Cm² flasks. Cloned cells are then frozen or injected into mice for ascites production.

What is claimed is:

1. An isolated nucleic acid comprising a nucleotide sequence that encodes the Proteus mirabilis adhesin polypeptide of FIG. 2.

2. An isolated nucleic acid comprising a nucleotide sequence that encodes amino acids 23-257 of FIG. 3.

3. An isolated nucleic acid comprising a nucleotide sequence that encodes amino acids 23-157 of FIG. 3.

4. A replicable expression vector comprising the nucleic acid of any one of claims 1, 2 or 3.

5. A replicable expression vector comprising a nucleic acid which encodes a polypeptide having the amino acid sequence shown in FIG. 2.

6. A microorganism or cell transformed by the nucleic acid of any one of claims 1, 2 or 3.

7. A microorganism or cell transformed by the vector of claim 4.

8. A microorganism or cell transformed by a nucleic acid which encodes a polypeptide having the amino acid sequence shown in FIG. 3.

9. A process of preparing a recombinant Proteus mirabilis adhesin polypeptide comprising cultivating the microorganism or cell of claim 6 for a time and under conditions sufficient to produce said polypeptides and recovering said adhesin polypeptide.

10. A vaccine for immunization of a vertebrate comprising an immunologically effective amount of an isolated Proteus mirabilis adhesin polypeptide or fragment thereof and a pharmaceutically acceptable carrier,

11. The vaccine of claim 10, wherein the polypeptide comprises an amino acid sequence of at least 20 amino acids and up to the entire amino acid sequence of the adhesin polypeptide.

12. The vaccine of claim 10, wherein the polypeptide comprises a 20-257 amino acid fragment of the adhesin polypeptide.

13. The vaccine of claim 10, wherein the polypeptide comprises a 23-157 amino acid fragment of the adhesin polypeptide.

14. An isolated Proteus mirabilis adhesin polypeptide comprising amino acids 1-275 of FIG. 3.

15. A purified antibody directed against a Proteus mirabilis adhesin polypeptide.

16. The antibody of claim 15, wherein said antibody is monoclonal or polyclonal.

17. A pharmaceutical composition comprising the antibody of claim 15.

18. A method of immunizing a mammalian subject against disease caused by Proteus mirabilis which comprises administering to the subject the vaccine of claim 10.

19. A method of preventing kidney stone formation caused by Proteus mirabilis which comprises administering to the subject the vaccine of claim 10.

20. A method of preventing bladder stone formation caused by Proteus mirabilis which comprises administering to the subject the vaccine of claim 10.

21. A method of detecting Proteus mirabilis infection comprising contacting a test sample with an antibody directed against an MrpH polypeptide, or an antigenic peptide thereof, for a time and under conditions sufficient to form an antigen-antibody complex and detecting said complex.

22. A pharmaceutical composition comprising a Proteus mirabilis adhesin polypeptide and a pharmaceutically acceptable carrier.

23. A pharmaceutical composition comprising a Proteus mirabilis adhesin polypeptide fragment and a pharmaceutically acceptable carrier.
